SEQUENCE LISTING (24 sequences)

SEQ ID NO: 1 - protein, 117 aa, synthetic construct [sequence omitted]

SEQ ID NO: 2 - protein, 108 aa, synthetic construct [sequence omitted]

SEQ ID NO: 3 - protein, 5 aa, synthetic construct
  source 1..5: note = HCDR1
NYWIA

SEQ ID NO: 4 - protein, 17 aa, synthetic construct
  source 1..17: note = HCDR3
IIYPGDSDIRYSPSFQG

SEQ ID NO: 5 - protein, 8 aa, synthetic construct
  source 1..8: note = HCDR3
HDIEGFDY

SEQ ID NO: 6 - protein, 12 aa, synthetic construct
  source 1..12: note = LCDR1
RASQSVSSSFFA

SEQ ID NO: 7 - protein, 7 aa, synthetic construct
  source 1..7: note = LCDR2
GASSRAT

SEQ ID NO: 8 - protein, 9 aa, synthetic construct
  source 1..9: note = LCDR3
QQYDSSAIT

SEQ ID NO: 9 - protein, 107 aa, synthetic construct [sequence omitted]

SEQ ID NO: 10 - protein, 330 aa, synthetic construct
  source 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 447 aa, synthetic construct
  source 1..447: note = Heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, synthetic construct
  source 1..215: note = Light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 80 aa, synthetic construct
  source 1..80: note = H15D10
EVQLVQSGAEVKKPGESLRISCKGSGYTFTNYWVAWVRQMPGKGLESMGIIYPGDSDTRYSPSFQGHVTISADKSISTAY

SEQ ID NO: 14 - protein, 108 aa, synthetic construct
  source 1..108: note = L8C3
DIQMTQSPSSLSASLGDRVTITCRASQNVGNYLNWYQQKPGKAPKLLIYRASNLASGVPSRFSGSGSGTDFTLTISSLQPEDFATYYCQQMEHAPPTFGQGTKVEIKR

SEQ ID NO: 15 - protein, 108 aa, synthetic construct
  source 1..108: note = L16C11
EIVLTQSPGTLSLSPGERATLSCRASQSVIGYYLAWYQQKPGQAPRLLIYSVSTLASGIPDRFSGSGSGTDFTLTISRLEPEDFAVYYCQQYYRFPITFGQGTKVEIK

SEQ ID NO: 16 - protein, 117 aa, synthetic construct
  source 1..117: note = H19B7
EVQLVQSGAEVKKPGESLRISCKGSGYTFTNYWMAWVRQMPGKGLESMGIIYPSDSDTRYSPSFQGHVTISADKSISTAYLQWSSLKASDTAMYYCARHDVEGYDYWGQGTLVTVSS

SEQ ID NO: 17 - protein, 119 aa, synthetic construct
  source 1..119: note = QX006N (VH)
EVQLVESGGGLVQPGGSLRLSCAASGFSLSSYYMTWVRQAPGKGLEWVSVINVYGGTYYASWAKGRFTISRDNSKNTLYLQMNSLRAEDTAVYYCAREDVAVYMAIDLWGQGTLVTVSS

SEQ ID NO: 18 - protein, 111 aa, synthetic construct
  source 1..111: note = QX006N (VL)
AIQMTQSPSSLSASVGDRVTITCQASQSISNQLSWYQQKPGKAPKLLIYDASSLASGVPSRFSGSRSGTKFTLTISSLQPEDFATYYCLGIYGDGADDGIAFGGGTKVEIK

SEQ ID NO: 19 - protein, 5 aa, synthetic construct
  source 1..5: note = QX006N (HCDR1)
SYYMT

SEQ ID NO: 20 - protein, 16 aa, synthetic construct
  source 1..16: note = QX006N (HCDR2)
VINVYGGTYYASWAKG

SEQ ID NO: 21 - protein, 11 aa, synthetic construct
  source 1..11: note = QX006N (HCDR3)
EDVAVYMAIDL

SEQ ID NO: 22 - protein, 11 aa, synthetic construct
  source 1..11: note = QX006N (LCDR1)
QASQSISNQLS

SEQ ID NO: 23 - protein, 7 aa, synthetic construct
  source 1..7: note = QX006N (LCDR2)
DASSLAS

SEQ ID NO: 24 - protein, 13 aa, synthetic construct
  source 1..13: note = QX006N (LCDR3)
LGIYGDGADDGIA

(19) United States
(12) Patent Application Publication
(10) Pub. No.: US 2026/0125482 A1
(43) Pub. Date: May 7, 2026

(54) TREATMENT OF LUPUS

(71) Applicant: ASTRAZENECA AB, Södertälje (SE)

(72) Inventors: Catharina LINDHOLM, Södertälje (SE); Gabriel ABREU, Södertälje (SE); Hussein AL-MOSSAWI, CAMBRIDGE (GB)

(21) Appl. No.: 19/117,553

(22) PCT Filed: Oct. 12, 2023
(86) PCT No.: PCT/EP2023/078291

§ 371 Date: Apr. 1, 2025

Related U.S. Application Data
(60) Provisional application No. 63/379,303, filed on Oct. 13, 2022.

(51) Int. Cl. C07K 16/28; A61K 9/00; A61K 39/00; A61K 39/215; A61P 37/06

(52) U.S. Cl. CPC C07K 16/2866; A61K 9/0019; A61K 39/215; A61P 37/06; A61K 2039/545; C07K 2317/21; C07K 2317/52; C07K 2317/565; C07K 2317/76

(57) ABSTRACT

The disclosure relates to methods and compositions for the treatment of systemic lupous erythematosus (SLE). Specifically, the disclosure relates to methods comprising administering to a subject a type I IFN receptor inhibitor.

### BACKGROUND

Anifrolumab (MEDI-546) is a human immunoglobulin G1 kappa (IgG1κ) monoclonal antibody (mAb) directed against subunit 1 of the type I interferon receptor (IFNAR1). It is composed of 2 identical light chains and 2 identical heavy chains, with an overall molecular weight of approximately 148 kDa. Anifrolumab inhibits binding of type I IFN to type I interferon receptor (IFNAR) and inhibits the biologic activity of all type I IFNs.

Systemic lupus erythematosus (SLE) is a chronic autoimmune disease characterized by multisystem involvement that can range from mild to life-threatening disease. Given the need for long-term treatment, it is important to assess the safety and efficacy of new therapies for SLE over an extended period. However, long-term open-label studies of biologic agents have left some questions remaining because of the lack of an appropriate control comparison group [1,2].

SLE disproportionately impacts populations most severely affected by COVID-19. Individuals with SLE are often heavily immunosuppressed and have a high comorbidity burden with multiple risk factors for more severe COVID-19. The causative agent of COVID-19 is SARS-Cov-2. Although previous analyses have evaluated outcomes of infection with SARS-Cov-2 in rheumatic diseases as a group, data on individuals with SLE are limited, and it remains unclear which risk factors are associated with worse COVID-19 outcomes in this population.

The effect of anifrolumab on SARS-CoV-2 infection or SARS-CoV-2 vaccination efficacy has not been assessed. Vaccination efficacy relies on stimulation of an immune response against the vaccine in the vaccinated individual. Inhibitors of type I IFN might therefore be expected to reduce SARS-CoV2 vaccination efficacy. SARS-CoV2 vaccination has been shown to be less effective in immunocompromised populations [3]. It has further been suggested that treatment with anifrolumab may increase the risk of COVID-19 related adverse events, and that SLE patients should be vaccinated against viral infection before receiving treatment with anifrolumab[4]. Treatment of patients with the biologic drug rituximab impairs the antibody response to immunization in patients with rheumatic disease for at least 6-9 months after an infusion the biologic [5].

The present invention solves one or more of the above-mentioned problems.

### SUMMARY

The present invention relates to a method for simultaneously treating systemic lupus erythematosus (SLE) and preventing or reducing the risk of SARS-CoV-2 infection in a subject in need thereof, the method comprising administering a type I IFN receptor (IFNAR1) inhibitor to the subject, and administering a therapeutically effective amount of a SARS-CoV-2 vaccine to the subject, wherein the method prevents or reduces the risk of SARS-CoV-2 infection in the subject following exposure of the subject to SARS-CoV-2, and wherein the method treats SLE in the subject.

The invention also relates to a method for preventing or reducing the risk of SARS-CoV-2 infection in a subject in need thereof, wherein the subject has an autoimmune disease and has been treated with a IFNAR1 inhibitor. The method comprises administering a therapeutically effective amount of a SARS-CoV-2 vaccine to the subject, wherein the method prevents or reduces the risk of SARS-CoV-2 infection in the subject following exposure of the subject to SARS-CoV-2.

The invention also relates to a method of treating SLE in a subject in need thereof, wherein the subject has been vaccinated against COVID-19 with a SARS-CoV-2 vaccine. The method comprises administering a therapeutically effective amount of a IFNAR1 inhibitor to the subject and treats SLE in the subject.

The invention also relates to pharmaceutical composition and injection devices for use in such methods.

The invention is supported inter alia by data, presented herein for the first time, from a randomized, placebo-controlled phase 3 extension trial of the long-term safety and tolerability of anifrolumab in SLE (NCT02794285). The data describe the first long-term placebo-controlled study in SLE or a biologic during the global COVID-19 pandemic. The data surprisingly show that patients treated with an IFNAR1 respond to vaccination with a SARS-CoV-2 vaccine. Vaccination of patients that are on long-term treatment with an IFNAR1 is shown to effective at preventing COVID-19 infection, COVID-19 pneumonia and COVID-19 associated death in the patients. The data further surprisingly show that COVID-19 vaccination does not effect the efficacy of treatment with a IFNAR1 inhibitor in patients with SLE.

### BRIEF DESCRIPTION OF FIGURES

FIG. 1: Patients included in the TULIP-1 or TULIP-2 trials and LTE study: treatment randomization and treatment group definitions

LTE, long-term extension. ^{a}There was an 8-week safety follow-up period. ^{b}Patients randomized to anifrolumab 150 mg were all in TULIP-1. ^{c}Patients were re-randomized to anifrolumab 300 mg or placebo for the LTE study.

FIG. 2: COVID-19 AEs during the pandemic

AE, adverse event; BBC, British Broadcasting Corporation; COVID-19, coronavirus disease 2019; LTE, long-term extension; SAE, serious adverse event; SARS-CoV-2, severe acute respiratory syndrome coronavirus 2; WHO, World Health Organization.

Date of vaccine introduction: Argentina 29 Dec. 2020; Australia 21 Feb. 2021; Bulgaria 30 Dec. 2020; Canada 14 Dec. 2020; Chile 21 Dec. 2020; Columbia 17 Feb. 2021; France 30 Dec. 2020; Germany 23 Dec. 2020; Hungary 23 Dec. 2020; Israel 16 Dec. 2020; Japan 17 Feb. 2021; Lithuania 23 Dec. 2020; Mexico 24 Dec. 2020; Peru 9 Feb. 2021; Poland 23 Dec. 2020; Republic of Korea 26 Feb. 2021; Romania 27 Jan. 2021; Russia 5 Dec. 2020; South Africa 17 Feb. 2021; Spain 30 Dec. 2020; Taiwan 22 Mar. 2021; Ukraine 24 Feb. 2021; The United Kingdom 21 Dec. 2020; United States of America 14 Dec. 2020.

Source for vaccine introduction dates: Russia (BBC News), Taiwan (Ministry of Foreign Aggairs, Replic of China [Taiwan]), other countries (WHO).

Patients with multiple events are represented only once, reporting the event of highest severity (death, SAE, and AE; COVID-19 pneumonia, COVID-19, SARS-CoV-2 test positive). One of the AEs of COVID-19 pneumonia was not considered an SAE and did not require in-patient hospitalization.

Circled points indicate that the event occurred after vaccination. One patient in the anifrolumab group received a single dose of the COVID-19 vaccine prior to the reported event.

FIG. 3: Change in mean SLEDAI-2K score from baseline to Week 216

FU, follow-up; LS, least squares; LTE, long-term extension; SE, standard error; SLEDAI-2K, Systemic Lupus Erythematosus Disease Activity Index 2000.

FIG. 4: Cumulative glucocorticoid dose standardized AUC over the 4-year study period for patients in the combined anifrolumab 300 mg or combined placebo group, excluding 3 patients from the combined anifrolumab 300 mg group who had glucocorticoid dose >40 mg/day at TULIP study baseline

AUC, area under the curve; GC, glucocorticoid; LTE, long-term extension; SE, standard error.

FIG. 5: Mean SLEDAI-2K score and mean GC dose standardized AUC for patients receiving combined anifrolumab 300 mg or placebo, by study year

AUC, area under the curve; GC, glucocorticoid; SE, standard error; SLEDAI-2K Systemic Lupus Erythematosus Disease Activity Index 2000. Note: Analysis excluded patients with glucocorticoid doses >40 mg/day at baseline. ^{a}Number of participants at risk for SLEDAI-2K was n=358 (combined anifrolumab 300 mg) and n=178 (combined placebo) for all time points.

FIG. 6: Percentage of patients by glucocorticoid dose group at each year during the TULIP trials and the extension study

A, anifrolumab; P, placebo; GC, glucocorticoid; LTE, long-term extension. Proportion of patients by GC dosage over time, excluding patients with GC dose >40 mg/day at baseline. Combined anifrolumab 300 mg includes patients randomized to anifrolumab 300 mg at the start of the TULIP study and continued on anifrolumab 300 mg in the extension study. Combined placebo includes patients randomized to placebo in either TULIP study and the extension study.

FIG. 7: Mean±SE change from baseline (LOCF) in SDI global score among patients with SDI ≥1 at Week 52

LOCF, last operation carried forward; LTE, long-term extension; SDI, SDI Systemic Lupus International Collaborating Clinics/American College of Rheumatology Damage Index; SE, standard error.

### DETAILED DESCRIPTION

### Methods of Treatment

The invention relates to a method for treating systemic lupus erythematosus (SLE) and for preventing or reducing the risk of SARS-CoV-2 infection in a subject in need thereof, the method comprising a) administering a type I IFN receptor (IFNAR1) inhibitor to the subject, and b) administering a therapeutically effective amount of a SARS-CoV-2 vaccine to the subject; wherein the method prevents or reduces the risk of SARS-CoV-2 infection in the subject following exposure of the subject to SARS-CoV-2, and wherein the method treats SLE in the subject.

The invention also relates to a method for preventing or reducing the risk of SARS-CoV-2 infection in a subject in need thereof, wherein the subject has an autoimmune disease and has been treated with a IFNAR1 inhibitor, the method comprising administering a therapeutically effective amount of a SARS-CoV-2 vaccine to the subject, and wherein the method prevents or reduces the risk of SARS-CoV-2 infection in the subject following exposure of the subject to SARS-CoV-2.

The invention also relates to a method for treating SLE in a subject in need thereof, wherein the subject has been vaccinated against COVID-19 with a SARS-CoV-2 vaccine, the method comprising administering a therapeutically effective amount of a IFNAR1 inhibitor to the subject, wherein the method treats SLE in the subject.

The invention also relates to pharmaceutical compositions and injection devices for use in the methods of the invention. The invention relates to a pharmaceutical composition for use a method of preventing or reducing the risk of SARS-CoV-2 infection in a subject, wherein the subject has an autoimmune disease and has been treated with a IFNAR1 inhibitor, wherein the pharmaceutical composition comprises a SARS-CoV-2 vaccine, the method comprising administering the pharmaceutical composition to the subject, wherein the pharmaceutical composition prevents or reduces the risk of SARS-CoV-2 infection in the subject following exposure of the subject to SARS-CoV-2. The invention also relates to a pharmaceutical composition for use in a method of treating SLE in a subject in need thereof, wherein the subject has been vaccinated against COVID-19 with a SARS-CoV-2 vaccine, wherein the pharmaceutical composition comprises a IFNAR1 inhibitor, the method comprising administering the pharmaceutical composition to the subject, wherein the method treats SLE in the subject. The autoimmune disease may be lupus nephritis, cutaneous lupus erythematosus, myositis or scleroderma.

### COVID-19 and SARS-CoV-2 Vaccine

Administration of the SARS-CoV-2 vaccine may prevent or reduce the risk of COVID-19 pneumonia in the subject. Administration of the SARS-CoV-2 vaccine may prevent or reduce the risk of COVID-19 associated death in the subject. Administration of the SARS-CoV-2 vaccine may prevent or reduce COVID-19 related adverse events in the subject.

The IFNAR1 inhibitor may be administered to the subject less than 1 month before the SARS-CoV-2 vaccine. The IFNAR1 inhibitor maybe administered on the same day as the SARS-CoV-2 vaccine.

The SARS-CoV-2 vaccine may be administered to the subject about 1 month or less following administration of the IFNAR1 inhibitor to the subject. The SARS-CoV-2 vaccine may be administered to the subject 1 month following administration of the IFNAR1 inhibitor to the subject. The SARS-CoV-2 vaccine may be administered to the subject about 6, 5-, 4-, 3- or 2-months following administration of the IFNAR1 inhibitor to the subject. The SARS-CoV-2 vaccine may be administered to the subject 1 month following administration of the IFNAR1 inhibitor to the subject. The SARS-CoV-2 vaccine may be administered to the subject about 4 weeks following administration of the IFNAR1 inhibitor to the subject. The SARS-CoV-2 vaccine may be administered to the subject about 3, 2 or 1 weeks following administration of the IFNAR1 inhibitor to the subject.

The method may comprise administering at least 2 doses of the SARS-CoV-2 vaccine to the subject. The 2 doses of the SARS-CoV-2 vaccine may be administered to the subject within a period of 21 to 28 days. The 2 doses of the SARS-CoV-2 vaccine may be administered to the subject within a period of 14 to 56 days.

The IFNAR1 inhibitor may have been administered to the subject less than 1 month before the SARS-CoV-2 vaccine is administered to the subject.

The subject may have been fully vaccinated against COVID-19. The subject may have received at least 2 doses of the SARS-CoV-2 vaccine. The 2 doses of the SARS-CoV-2 vaccine may have been administered to the subject within a period of 21 to 28 days. The 2 doses of the SARS-CoV-2 vaccine may have been administered to the subject within a period of 14 to 56 days. The subject may have been vaccinated against COVID-19 6 months or less before administration of the IFNAR1 inhibitor.

The SARS-CoV-2 vaccine may be selected from the group consisting of AZD1222, mRNA-1273, or BNT162b2 Tozinameran or combinations thereof. The SARS-CoV-2 vaccine may be selected from the group consisting of SARS-CoV-2 vaccines include for following: intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (InCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), SARS-Cov-2 coronavirus vaccine NVX-CoV2373 (Novavax), an intramuscular vaccine INO-4700 (GLS-5300) (Inovio Pharma and GeneOne Life Science), and combinations thereof.

### SARS-CoV-2 Vaccine

### IFNAR1 Inhibitor

A “type I interferon receptor inhibitor” refers to a molecule that is antagonistic for the receptor of type I interferon ligands such as interferon-α and interferon-β. Such inhibitors, subsequent to administration to a patient, preferably provide a reduction in the expression of at least 1 (preferably at least 4) pharmacodynamic (PD) marker genes selected from the group consisting of IFI6, RSAD2, IFI44, IFI44L, IFI27, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IFI27, IFI44, IFI44L, and RSAD2. The “type I interferon receptor” is preferably interferon-α/β receptor (IFNAR).

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-α/β receptor (IFNAR) antagonist. The type I interferon receptor inhibitor may be an antibody or antigen-binding fragment thereof that inhibits type I IFN activity. Additionally or alternatively, the type I interferon receptor inhibitor may be a small molecule inhibitor of a type I interferon receptor (e.g. for pharmacological inhibition of type I interferon receptor activity).

The IFNAR1 inhibitor may be a human monoclonal antibody specific for IFNAR1. The IFNAR1 inhibitor may be a modified IgG1 class human monoclonal antibody specific for IFNAR1.

The antibody may comprise a heavy chain variable region complementarity determining region 1 (HCDR1) comprising the amino acid sequence of SEQ ID NO: 3. The antibody may comprise a heavy chain variable region complementarity determining region 2 (HCDR2) comprising the amino acid sequence of SEQ ID NO: 4. The antibody may comprise a heavy chain variable region complementarity determining region 3 (HCDR3) comprising the amino acid sequence of SEQ ID NO: 5. The antibody may comprise a light chain variable region complementarity determining region 1 (LCDR1) comprising the amino acid sequence SEQ ID NO: The antibody may comprise a light chain variable region complementarity determining region 2 (LCDR2) comprising the amino acid sequence SEQ ID NO: 7. The antibody may comprise a light chain variable region complementarity determining region 3 (LCDR3) comprising the amino acid sequence SEQ ID NO: 8.

The antibody may comprise a human heavy chain variable region comprising the amino acid sequence of SEQ ID NO: 1. The antibody may comprise a human light chain variable region comprising the amino acid sequence of SEQ ID NO: 2. The antibody may comprise a human light chain constant region comprising the amino acid sequence of SEQ ID NO: 9. The antibody may comprise a human heavy chain constant region comprising the amino acid sequence of SEQ ID NO: 10. The antibody may comprise in the Fc region an amino acid substitution of L234F, as numbered by the EU index as set forth in Kabat and wherein said antibody exhibits reduced affinity for at least one Fc ligand compared to an unmodified antibody. The antibody may comprise a human heavy chain comprising the amino acid sequence of SEQ ID NO: 11. The antibody may comprise a human light chain comprising the amino acid sequence of SEQ ID NO: 12.

The antibody may comprise: (a) a heavy chain variable region complementarity determining region 1 (HCDR1) comprising the amino acid sequence of SEQ ID NO: 3; (b) a heavy chain variable region complementarity determining region 2 (HCDR2) comprising the amino acid sequence of SEQ ID NO: 4; c) a heavy chain variable region complementarity determining region 3 (HCDR3) comprising the amino acid sequence of SEQ ID NO: 5; (d) a light chain variable region complementarity determining region 1 (LCDR1) comprising the amino acid sequence SEQ ID NO: 6; (b) a light chain variable region complementarity determining region 2 (LCDR2) comprising the amino acid sequence SEQ ID NO: 7; c) a light chain variable region complementarity determining region 3 (LCDR3) comprising the amino acid sequence SEQ ID NO: 8.

The antibody may comprise (a) a human heavy chain comprising the amino acid sequence of SEQ ID NO: 11; and (b) a human light chain comprising the amino acid sequence of SEQ ID NO: 12.

The IFNAR1 inhibitor may be anifrolumab or a functional variant thereof.

### Doses and Methods of Administration of the IFNAR1 Inhibitor

The IFNAR1 inhibitor may be administered intravenously or subcutaneously. The IFNAR1 inhibitor may be administered a dose of 120 mg to 1000 mg. The IFNAR1 inhibitor may be administered to the subject intravenously at a dose of about 300 mg every 4 weeks (Q4W). IFNAR1 inhibitor may be administered to the subject intravenously at a dose of about 900 mg every 4 weeks (Q4W). The IFNAR1 inhibitor may be administered to the subject subcutaneously at a dose of about 120 mg every week.

The IFNAR1 inhibitor may have been administered to the subject intravenously or subcutaneously. The IFNAR1 inhibitor may have been administered to the subject at a dose of 120 mg to 1000 mg.

The IFNAR1 inhibitor may have been administered to the subject intravenously at a dose of about 300 mg every 4 weeks (Q4W). The IFNAR1 inhibitor may have been administered to the subject intravenously at a dose of about 900 mg every 4 weeks (Q4W). The IFNAR inhibitor may have been administered to the subject subcutaneously at a dose of about 120 mg every week.

The method may comprise administering an intravenous dose of anifrolumab or the functional variant thereof to the subject. The intravenous dose may be ≥300 mg anifrolumab or the functional variant thereof. The intravenous dose may be 51000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be 5135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 m. The subcutaneous dose may have a volume of about 0.8 ml.

The dosage regimen for the IFNAR1 inhibitor may comprise a first intensive regime (IR) comprising ×3 intravenous 900 mg doses Q4W, followed by basic regime (BR) of a) weekly subcutaneous 120 mg dose, or b) an intravenous 300 mg dose Q4W (or a mixture of a) and b)). The IFNAR1 inhibitor may be anifrolumab or a functional variant thereof. The dosage regimen may comprise a first intensive regime (IR) comprising ×6 intravenous 900 mg doses Q4W, followed by basic regime (BR) of a) weekly subcutaneous 120 mg dose, or b) an intravenous 300 mg dose Q4W (or a mixture of a) and b)). The dosage regimen may comprise a first intensive regime (IR) comprising ×6 subcutaneous 1150 mg doses Q4W, followed by basic regime (BR) of a) weekly subcutaneous 120 mg dose, or b) an intravenous 300 mg dose Q4W (or a mixture of a) and b)). The IFNAR1 inhibitor may be anifrolumab or a functional variant thereof.

The method may comprise administering intravenously an intravenous dose of an IFNAR1 inhibitor (e.g. anifrolumab or the functional variant thereof to the subject). The intravenous dose may be ≥300 mg of an IFNAR1 inhibitor (e.g. anifrolumab or the functional variant thereof). The intravenous dose may be 51000 mg of an IFNAR1 inhibitor (e.g. anifrolumab or the functional variant thereof). The intravenous dose may be 900 mg to 1000 mg of an IFNAR1 inhibitor (e.g. anifrolumab or the functional variant thereof). The intravenous dose may be >300 mg of an IFNAR1 inhibitor (e.g. anifrolumab or the functional variant thereof). The intravenous dose may be about 300 mg, about 900 mg or 1000 mg of an IFNAR1 inhibitor (e.g. anifrolumab or the functional variant thereof). The intravenous dose may be 300 mg, 900 mg or 1000 mg of an IFNAR1 inhibitor (e.g. anifrolumab or the functional variant thereof). The intravenous dose may be administered about every four weeks (Q4W). The intravenous dose may be administered about every month. A dose of 300 mg IV dose may be administered using an infusion pump over a minimum of 30 minutes. A dose of 900 mg IV dose may be administered using an infusion pump over a minimum of 60 minutes. An anifrolumab 300 mg IV dose may be supplied as a 2 ml vial, at a concentration of 150 mg/mL.

The method may comprise administering subcutaneously a subcutaneous dose of anifrolumab or the functional variant thereof. The subcutaneous dose may be administered after, before or in between intravenous administration of the intravenous dose. The subcutaneous dose may be >105 mg and <150 mg anifrolumab or the functional variant thereof. The subcutaneous dose may be 5135 mg anifrolumab or the functional variant thereof. The subcutaneous dose may be about 120 mg anifrolumab or the functional variant thereof. The subcutaneous dose may be administered in a single administration step. The subcutaneous dose may be administered at intervals of 6-8 days. The subcutaneous dose may be administered once per week. The subcutaneous dose may have a volume of 0.5 to 1 ml. The subcutaneous dose may have a volume of 0.5 to 1.0 ml. The subcutaneous dose may have a volume of about 0.8 ml. The subcutaneous dose may have a volume of 0.8 ml. The subcutaneous dose may about 1150 mg (e.g. 1155 or 1150 mg) anifrolumab or the functional variant thereof. The subcutaneous dose may have a volume of about 8 ml. The subcutaneous dose may have a volume of about 7.7 ml.

The method may comprise administering to the subject a first dose of a IFNAR1 inhibitor, followed by a second dose of the IFNAR1 inhibitor, wherein the first dose is higher than the second dose. The first dose may be administered intravenously. The first dose may be >300 mg. The first dose may be <1000 mg. The first dose may be about 900 mg. The first dose may be administered Q4W. The first dose may be administered to the subject 3 times before the second dose is administered to the subject. The first dose may be administered to the subject 6 times before the second dose is administered to the subject. The first dose may be administered every 4 weeks for 12 weeks before the second dose is administered. The first dose may be administered every 4 weeks for 24 weeks before the second dose is administered. The first dose may be administered subcutaneous. The first dose may be about 1150 mg or 1150 mg. The first dose may be administered Q4W. The first dose may be administered to the subject 3 times before the second dose is administered to the subject. The first dose may be administered to the subject 6 times before the second dose is administered to the subject. The first dose may be administered every 4 weeks for 12 weeks before the second dose is administered. The first dose may be administered every 4 weeks for 24 weeks before the second dose is administered. The intravenous dose may be administered as part of an intensive dosage regime (IR), wherein the total dose of the IFNAR1 inhibitor administered during the IR is 2.7 to 81 g, optionally 72.9 g, over a 12 to 24 week period. The IR may comprise administration of a SC dose of the IFNAR1 inhibitor that is equivalent to an IV dose of 900 to 1000 mg Q4W.

The second dose may be administered subcutaneously. The second dose may be >105 mg and ≤135 mg and administer subcutaneously. The second dose may be about 120 mg and administer subcutaneously. The second dose may be administered once per week.

The second dose may be administered intravenously. The second dose may be administered every month. The second dose may be administered Q4W. The second dose may be ≥300 mg. The second dose may be ≤1000 mg and administered intravenously. The second dose may be about 300 mg and administered intravenously. The dose may be about 900 mg and administered intravenously Q4W, wherein the second dose is about 120 mg administered subcutaneously QW. The dose may be about 900 mg and administered intravenously Q4W, wherein the second dose is about 300 mg administered intravenously Q4W, optionally wherein the first dose is administered to the subject at least 3 times before the second dose is administered to the patient, optionally wherein the first dose is administered to the subject at least 6 times before the second dose is administered to the patient. The second dose may be administered for at least a year.

The method may comprise administering a unit dose or pharmaceutical composition comprising about 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 305, 310, 800, 805, 810, 820, 825, 830, 835, 840, 845, 850, 855, 860, 865, 870, 875, 880, 885, 890, 895, 890, 900, 905, 910, 915, 920, 925, 930, 935, 940, 945, 950, 955, 960, 965, 970, 975, 980, 985, 990, 1000, 1050, 1010, 1020, 1025, 1030, 1035, 1040, 1045, 1050, 1055, 1060, or 1065 mg of an IFNAR1 inhibitor (e.g. anifrolumab or the functional variant thereof).

The subject may be a type I interferon stimulated gene signature (IFNGS)-test high patient pre-treatment. The method may comprise identifying the subject as IFNGS-test high patient pre-treatment.

Many patients with SLE receive corticosteroids (glucocorticoids, oral corticosteroids, OCS). However, corticosteroids are associated with organ damage. Anifrolumab permits tapering of the corticosteroids (glucocorticoids) in SLE patients (steroid sparing). The method of treatment or method may comprise administering a corticosteroid to the subject, optionally wherein the corticosteroid is an oral corticosteroid. The method may comprise tapering dose of corticosteroids administered to the subject (steroid sparing). The method may comprise administering a first dose of the corticosteroid and subsequently administering a second dose of the corticosteroid, wherein the second dose of the corticosteroid is lower than the first dose of the corticosteroid. The second dose of the corticosteroid may be about a 7.5 mg prednisone-equivalent dose or less. The second dose of the corticosteroid may be a 5 mg prednisone-equivalent dose or less. The method or method of treatment may comprise administrating the second dose of the corticosteroid once per day. The first dose of the corticosteroid may be about a 10 mg prednisone-equivalent dose. The method may comprise tapering the dose of corticosteroid administered to the patient from 10 mg or more per day to less than 10 mg per day. The method or method of treatment may comprise administering the second dose of the corticosteroid once per day. The method may permit administration of a reduced dose of corticosteroids that is sustained for weeks. The second dose of the corticosteroid may be administered for at least 24 weeks. The second dose of the corticosteroid may be administered for at least 28 weeks.

### Steroid Sparing

The method may comprise steroid sparing in the subject, wherein the dose of the steroid administered to the subject is tapered from a pre-sparing dose at baseline to a post-sparing dose. The post-sparing dose may be ≤7.5 mg/day prednisone or prednisone equivalent dose. The pre-sparing dose may be 20 mg/day prednisone or prednisone equivalent dose. The steroid may comprise a glucocorticoid. The steroid may comprise an oral glucocorticoid. The steroid may be selected from the group consisting of hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof. The steroid may be prednisone.

### The Subject

The subject may be a human subject. The subject may be an adult. The subject may be a patient with an elevated type I IFN gene signature. The subject may be a type I interferon stimulated gene signature (IFNGS)-test high patient pre-administration with the dose or unit dose. The subject may have elevated of the genes IFI27, IFI44, IFI44L, and RSAD2 in the whole blood. The method may comprise identifying the subject as IFNGS-test high patient pre-treatment with the dose or unit dose. The method may comprise measuring the expression of the genes IFI27, IFI44, IFI44L, and RSAD2 in the whole blood of the subject. The method may comprise measuring the expression of the genes IFI27, IFI44, IFI44L, and RSAD2 in the whole blood of the subject by RT-PCR.

The subject may have moderate to severe SLE pre-treatment with the IFNAR1 inhibitor. Pretreatment with the IFNAR1 inhibitor, the subject may be refractive to treatment with the one more immunomodulators or has relapsed during or following treatment with the one or more immunomodulators. Pretreatment with the IFNAR1 inhibitor the subject may have a SLEDAI-2K score of ≥10 (at least). Pretreatment with the IFNAR1 inhibitor, the subject may have a CLASI activity score of ≥10 (at least 10). Pretreatment with the IFNAR1 inhibitor, the subject may have a swollen and tender joint count of ≥6.

The subject may have moderate to severe SLE as defined by the ACR Classification Criteria for SLE (ACR 1997 [10] and/or EULAR/ACR 2019 [11]).

### Pharmaceutical Composition

The invention also relates to a pharmaceutical composition for use in a method of treating CLE in a subject, the method comprising subcutaneously administering the pharmaceutical composition to a subject, wherein the pharmaceutical composition comprises a dose of anifrolumab or functional variant thereof, wherein the dose is >105 mg and <150 mg. The dose of anifrolumab of the functional variant thereof may be a unit dose (unit dose form, pharmaceutical unit dose form, pharmaceutical unit dose). Functional anifrolumab variants include antigen-binding fragments of anifrolumab and antibody and immunoglobulin derivatives of anifrolumab.

In another aspect the invention relates to a pharmaceutical composition for use in a method of treating SLE in a subject, the method comprising subcutaneously administering the pharmaceutical composition to the subject, wherein the pharmaceutical composition comprises a dose of anifrolumab or functional variant thereof, wherein administering the pharmaceutical composition every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is about equivalent to the plasma concentration provided by intravenous administration of 400 mg of anifrolumab or the functional variant thereof every 4 weeks. The dose may be <150 mg (i.e. less than 150 mg) anifrolumab or the functional variant thereof. The dose may be >105 mg (i.e. more than 105 mg) anifrolumab or the functional variant thereof. The dose may be ≤135 mg (i.e. 135 mg or less) anifrolumab or the functional variant thereof. The dose may be about 120 mg anifrolumab or the functional variant thereof. The dose may be 120 mg anifrolumab or the functional variant thereof.

The pharmaceutical composition may be administered at intervals of 6-8 days. The pharmaceutical composition may be administered once per week (QW). The pharmaceutical composition may be administered in a single administration step. The dose may be 120 mg anifrolumab or the functional variant thereof, and the method of treatment may comprise administering the dose in a single administration step once per week (QW). The pharmaceutical composition may be administered once per week for at least about 4 weeks. The pharmaceutical composition may be administered once per week for at least about 8 weeks. The dose or unit dose may be administered once per week for at least about 12 weeks. The pharmaceutical composition may be administered once per week for at least about 16 weeks. The pharmaceutical composition may be administered once per week for at least about 20 weeks. The pharmaceutical composition may be administered once per week for at least about 24 weeks. The pharmaceutical composition may be administered once per week for at least about 28 weeks. The pharmaceutical composition may be administered once per week for at least about 32 weeks. The pharmaceutical composition may be administered once per week for about 8 weeks. The pharmaceutical composition may have a volume permitted it suitable delivery in a single subcutaneous administration step. The pharmaceutical composition may have a volume of about 0.5 to about 1 ml. The pharmaceutical composition may have a volume of less than 1 ml. The pharmaceutical composition may have a volume of about 0.8 ml.

Administration of the pharmaceutical composition may provide a plasma concentration of anifrolumab or the functional variant thereof in the patient of ≥10 μg (i.e. 10 μg or more) anifrolumab or the functional variant thereof per ml of plasma (i.e. a plasma concentration of ≥10 μg/ml). Administration of the pharmaceutical composition may provide a plasma concentration of anifrolumab or the functional variant thereof in the subject of about 10-100 μg/ml. Administration of the pharmaceutical composition may provide a plasma concentration of anifrolumab or the functional variant thereof in the subject of about 20-80 μg/ml. Administration of the pharmaceutical composition may provide a plasma concentration of anifrolumab or the functional variant thereof in the subject of about 30-70 μg/ml. Administration of the pharmaceutical composition may provide a trough concentration of anifrolumab or the functional variant thereof in the subject of ≥20 μg/ml (i.e. 20 μg/ml or more). Administration of the pharmaceutical composition may provide a trough concentration of anifrolumab or the functional variant thereof in the subject of ≥30 μg/ml (i.e. 30 μg/ml or more). Administration of the pharmaceutical composition may provide a trough concentration of anifrolumab or the functional variant thereof in the subject of ≥40 μg/ml (i.e. 40 μg/ml or more). Administration of the pharmaceutical composition may provide a trough concentration of anifrolumab or the functional variant thereof in the subject of about 20-100 μg/ml. Administration of the pharmaceutical composition may provide a trough concentration of anifrolumab or the functional variant thereof in the subject of about 30-80 μg/ml. Administration of the pharmaceutical composition may provide a trough concentration of anifrolumab or the functional variant thereof in the subject of about 40-70 μg/ml.

The pharmaceutical composition may provide a therapeutic effect in the subject that is at least equivalent to a therapeutic effect provided by administration of an intravenous dose of 300 mg anifrolumab or the functional variant thereof administered once every (Q4W). The pharmaceutical composition may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

### Device

The invention also relates to an injection device comprising the pharmaceutical composition for use in the methods of the invention of the invention.

The pharmaceutical in the injection device may comprise >105 mg (i.e. more than 105 mg) and <150 mg (i.e. less than 150 mg) anifrolumab or a functional variant thereof. The pharmaceutical composition in the injection device may comprise about 120 mg anifrolumab or the functional variant thereof. The pharmaceutical composition in the injection device may comprise 120 mg anifrolumab or the functional variant thereof. The concentration of anifrolumab or the functional variant thereof in the pharmaceutical composition in the injection device may be 150 mg/ml. The volume of the pharmaceutical composition in the injection device may be at least about 0.8 ml. The volume of the pharmaceutical composition may be about 0.8 ml.

The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/mL anifrolumab or the functional variant thereof. The pharmaceutical composition in the injection device may comprise 50 mM lysine HCl. The pharmaceutical composition may comprise 130 mM trehalose dihydrate. The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/mL anifrolumab or the functional variant thereof. The pharmaceutical composition may comprise 50 mM lysine HCl. The pharmaceutical composition in the injection device may comprise 130 mM trehalose dihydrate. The pharmaceutical composition in the injection device may comprise 0.05% polysorbate 80. The pharmaceutical composition in the injection device may comprise 25 mM histidine/histidine HCl. The pharmaceutical composition in the injection device may comprise 150 mg/mL anifrolumab or the functional variant thereof, 50 mM lysine HCl, 130 mM trehalose dihydrate, 0.05% polysorbate 80 and 25 mM histidine/histidine HCl.

In another aspect the invention relates to an injection device comprising a unit dose for use in the methods of the invention. The unit dose may comprise >105 mg (i.e. at least 105 mg) and ≤150 mg (i.e. less than 150 mg) anifrolumab or a functional variant thereof. The unit dose may comprise 5135 mg (i.e. 135 mg or less) anifrolumab or the functional variant thereof. The unit dose may comprise about 120 mg anifrolumab or the functional variant thereof. The unit dose in the injection device may comprise 120 mg anifrolumab or the functional variant thereof. The unit dose in the injection device may consist essentially of >105 mg and <150 mg anifrolumab or the functional variant thereof. The unit dose in the injection device may consist essentially of ≤135 mg anifrolumab or the functional variant thereof. The unit dose in the injection device may consist essentially of about 120 mg anifrolumab or the or the functional variant thereof. The concentration of anifrolumab or the functional variant thereof in the unit dose in the injection device may be about 150 mg/ml. The volume of the unit dose in the injection device may be less than 1 ml. The unit dose in the injection device may have a volume of about 0.5 to about 1 ml. The concentration of the unit dose may be about 0.8 ml. The volume of the unit dose may be 0.8 ml. The unit dose in the injection device may comprise a formulation of about 150 to 200 mg/ml anifrolumab or the functional variant thereof, about 25 to 150 mM of lysine sale and an uncharged excipient. The unit dose in the injection device may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

The injection device may be a pre-filled syringe (PFS). The injection device may be an accessorized pre-filed syringe (AFPS). The injection device may be an auto-injector (AI).

### Kit

In another aspect the invention relates to a kit comprising a unit dose of the invention and instructions for use, wherein the instructions for use comprise instructions for subcutaneous administration of the IFNAR1 inhibitor to a subject.

In another aspect the invention relates to a kit comprising the pharmaceutical composition for the use of the invention, wherein the instructions for use comprise instructions for subcutaneous administration of the pharmaceutical composition to a subject.

In another aspect the invention relates to a kit comprising the injection device of any of the invention, and instructions for use, wherein the instruction for use comprise instructions for use of the injection device to subcutaneously administer the unit dose or pharmaceutical composition to the subject. The kit may comprise a pharmaceutical composition comprising a SARS-CoV-2 vaccine.

The kit of the invention may comprise packaging, wherein the packaging is adapted to hold the injection device and the instructions for use. The instructions for use may be attached to the injection device. The instruction for use may comprise instructions for administration of >105 mg and <150 mg anifrolumab or functional variant thereof. The instruction for use may comprise instructions for administration of ≤135 mg anifrolumab or the functional variant thereof. The instruction for use may comprise instructions for administration of 120 mg anifrolumab or the functional variant thereof. The instruction for use may comprise instructions for administration of 120 mg anifrolumab or the functional variant thereof every 4 weeks. The instructions for use may define the subject as having a type I IFN mediated disease. The instructions for use may define the subject as having an autoimmune disease. The instructions may define the subject as having SLE. The instructions may define the subject as having moderate to severe SLE. The instructions for use may be written instructions.

The instructions for use may specify that the injection device, unit dose and/or pharmaceutical composition are for use in the treatment of SLE or for use according to the methods of the invention. The instructions for use comprise instructions for administration of 120 mg anifrolumab or the functional variant thereof every week. The instructions for use may specify that the unit dose or pharmaceutical composition of the invention are for use in the treatment of a subject that may have received a SARS-CoV-2 vaccine. The instruction for use may specify that the unit dose or pharmaceutical composition of the invention is for use in any of the method of the invention. The instructions for use may specify that the method of the invention has been demonstrated in a phase III clinical trial.

### Formulations

The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

Stable formulations suitable for administration to subjects and comprising anifrolumab are described in detail in U.S. patent Ser. No. 10/125,195 B1, which is incorporated herein in its in entirety.

### Definitions

### IFNAR Inhibitors

### Anifrolumab

Anifrolumab (MEDI-546, anifro, ANI) is a human immunoglobulin G1 kappa (IgG1K) monoclonal antibody (mAb) directed against subunit 1 of the type I interferon receptor (IFNAR1). Anifrolumab downregulates IFNAR signaling and suppresses expression of IFN-inducible genes. Disclosures related to anifrolumab can be found in U.S. Pat. Nos. 7,662,381 and 9,988,459, which are incorporated herein by reference in their entirety. Sequence information for anifrolumab is provided in Table 1.

TABLE 1
Anifrolumab Sequences

| | |
|---|---|
| Anifrolumab VH | EVQLVQSGAEVKKPGESLKISCKGSGYIFT |
| (SEQ ID NO: 1) | NYWIAWVRQMPGKGLESMGIIYPGDSDIRY |
| | SPSFQGQVTISADKSITTAYLQWSSLKASD |
| | TAMYYCARHDIEGFDYWGRGTLVTVSS |
| Anifrolumab VL | EIVLTQSPGTLSLSPGERATLSCRASQSVS |
| (SEQ ID NO: 2) | SSFFAWYQQKPGQAPRLLIYGASSRATGIP |
| | DRLSGSGSGTDFTLTITRLEPEDFAVYYCQ |
| | QYDSSAITFGQGTRLEIK |
| HCDR1 | NYWIA |
| (SEQ ID NO: 3) | |
| HCDR2 | IIYPGDSDIRYSPSFQG |
| (SEQ ID NO: 4) | |
| HCDR3 | HDIEGFDY |
| (SEQ ID NO: 5) | |
| LCDR1 | RASQSVSSSFFA |
| (SEQ ID NO: 6) | |
| LCDR2 | GASSRAT |
| (SEQ ID NO: 7) | |
| LCDR3 | QQYDSSAIT |
| (SEQ ID NO: 8) | |
| Light chain | RTVAAPSVFIFPPSDEQLKSGTASVVCLLN |
| constant region | NFYPREAKVQWKVDNALQSGNSQESVTEQD |
| (SEQ ID NO: 9) | SKDSTYSLSSTLTLSKADYEKHKVYACEVT |
| | HQGLSSPVTKSFNRGEC |
| Heavy chain | ASTKGPSVFPLAPSSKSTSGGTAALGCLVK |
| constant region | DYFPEPVTVSWNSGALTSGVHTFPAVLQSS |
| (SEQ ID NO: 10) | GLYSLSSVVTVPSSSLGTQTYICNVNHKPS |
| | NTKVDKRVEPKSCDKTHTCPPCPAPEFEGG |
| | PSVFLFPPKPKDTLMISRTPEVTCVVVDVS |
| | HEDPEVKFNWYVDGVEVHNAKTKPREEQYN |
| | STYRVVSVLTVLHQDWLNGKEYKCKVSNKA |
| | LPASIEKTISKAKGQPREPQVYTLPPSREE |
| | MTKNQVSLTCLVKGFYPSDIAVEWESNGQP |
| | ENNYKTTPPVLDSDGSFFLYSKLTVDKSRW |
| | QQGNVFSCSVMHEALHNHYTQKSLSLSPGK |
| Heavy chain | EVQLVQSGAEVKKPGESLKISCKGSGYIFT |
| (SEQ ID NO: 11) | NYWIAWVRQMPGKGLESMGIIYPGDSDIRY |
| | SPSFQGQVTISADKSITTAYLQWSSLKASD |
| | TAMYYCARHDIEGFDYWGRGTLVTVSSAST |
| | KGPSVFPLAPSSKSTSGGTAALGCLVKDYF |
| | PEPVTVSWNSGALTSGVHTFPAVLQSSGLY |
| | SLSSVVTVPSSSLGTQTYICNVNHKPSNTK |
| | VDKRVEPKSCDKTHTCPPCPAPEFEGGPSV |
| | FLFPPKPKDTLMISRTPEVTCVVVDVSHED |
| | PEVKFNWYVDGVEVHNAKTKPREEQYNSTY |
| | RVVSVLTVLHQDWLNGKEYKCKVSNKALPA |
| | SIEKTISKAKGQPREPQVYTLPPSREEMTK |
| | NQVSLTCLVKGFYPSDIAVEWESNGQPENN |
| | YKTTPPVLDSDGSFFLYSKLTVDKSRWQQG |
| | NVFSCSVMHEALHNHYTQKSLSLSPGK |
| Light chain | EIVLTQSPGTLSLSPGERATLSCRASQSVS |
| (SEQ ID NO: 12) | SSFFAWYQQKPGQAPRLLIYGASSRATGIP |
| | DRLSGSGSGTDFTLTITRLEPEDFAVYYCQ |
| | QYDSSAITFGQGTRLEIKRTVAAPSVFIFP |
| | PSDEQLKSGTASVVCLLNNFYPREAKVQWK |
| | VDNALQSGNSQESVTEQDSKDSTYSLSSTL |
| | TLSKADYEKHKVYACEVTHQGLSSPVTKSF |
| | NRGEC |

Anifrolumab is an immunoglobulin comprising an HCDR1, HCDR2 and HCDR3 of SEQ ID NO: 3, SEQ ID NO: 4, and SEQ ID NO: 5, respectively (or functional variant thereof); and an LCDR1, LCDR2 and LCDR3 of SEQ ID NO: 6, SEQ ID NO: 7, and SEQ ID NO: 8, respectively (or functional variant thereof). Anifrolumab is an immunoglobulin comprising a VH of SEQ ID NO: 1 and a VL of SEQ ID NO: 2.

The constant region of anifrolumab has been modified such that anifrolumab exhibits reduced affinity for at least one Fc ligand compared to an unmodified antibody. Anifrolumab is a modified IgG class monoclonal antibody specific for IFNAR1 comprising in the Fc region an amino acid substitution of L234F, as numbered by the EU index as set forth in Kabat (1991, NIH Publication 91-3242, National Technical Information Service, Springfield, Va.). Anifrolumab is a modified IgG class monoclonal antibody specific for IFNAR1 comprising in the Fc region an amino acid substitution of L234F, L235E and/or P331S, as numbered by the EU index as set forth in Kabat (1991, NIH Publication 91-3242, National Technical Information Service, Springfield, Va.). Anifrolumab is an antibody comprising a light chain constant region of SEQ ID NO: 9. Anifrolumab is an antibody comprising a heavy chain constant region of SEQ ID NO: 10. Anifrolumab is an antibody comprising a light chain constant region of SEQ ID NO: 9 and a heavy chain constant region of SEQ ID NO: 10. Anifrolumab is an antibody comprising a heavy chain of SEQ ID NO: 11. Anifrolumab is an antibody comprising a light chain of SEQ ID NO: 12. Anifrolumab is an antibody comprising a heavy chain of SEQ ID NO: 11 and a light chain of SEQ ID NO: 12.

Functional variants of anifrolumab are sequence variants that perform the same function as anifrolumab. Functional variants of anifrolumab are variants that bind the same target as anifrolumab and have the same effector function as anifrolumab. Functional anifrolumab variants include antigen-binding fragments of anifrolumab and antibody and immunoglobulin derivatives of anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

For example, a variant of the reference (anifrolumab) antibody may comprise: a heavy chain CDR1 having at most 2 amino acid differences when compared to SEQ ID NO: 3; a heavy chain CDR2 having at most 2 amino acid differences when compared to SEQ ID NO: 4; a heavy chain CDR3 having at most 2 amino acid differences when compared to SEQ ID NO: 5; a light chain CDR1 having at most 2 amino acid differences when compared to SEQ ID NO: 6; a light chain CDR2 having at most 2 amino acid differences when compared to SEQ ID NO: 7; and a light chain CDR3 having at most 2 amino acid differences when compared to SEQ ID NO: 8; wherein the variant antibody binds to the target of anifrolumab (e.g. IFNAR) and preferably with the same affinity.

A variant of the reference (anifrolumab) antibody may comprise: a heavy chain CDR1 having at most 1 amino acid difference when compared to SEQ ID NO: 3; a heavy chain CDR2 having at most 1 amino acid difference when compared to SEQ ID NO: 4; a heavy chain CDR3 having at most 1 amino acid difference when compared to SEQ ID NO: 5; a light chain CDR1 having at most 1 amino acid differences when compared to SEQ ID NO: 6; a light chain CDR2 having at most 1 amino acid difference when compared to SEQ ID NO: 7; and a light chain CDR3 having at most 1 amino acid difference when compared to SEQ ID NO: 8; wherein the variant antibody binds to the target of anifrolumab (e.g. IFNAR) optionally with the same affinity.

A variant antibody may have at most 5, 4 or 3 amino acid differences total in the CDRs thereof when compared to a corresponding reference (anifrolumab) antibody, with the proviso that there is at most 2 (optionally at most 1) amino acid differences per CDR. A variant antibody may have at most 2 (optionally at most 1) amino acid differences total in the CDRs thereof when compared to a corresponding reference (anifrolumab) antibody, with the proviso that there is at most 2 amino acid differences per CDR. A variant antibody may have at most 2 (optionally at most 1) amino acid differences total in the CDRs thereof when compared to a corresponding reference (anifrolumab) antibody, with the proviso that there is at most 1 amino acid difference per CDR.

A variant antibody may have at most 5, 4 or 3 amino acid differences total in the framework regions thereof when compared to a corresponding reference (anifrolumab) antibody, with the proviso that there is at most 2 (optionally at most 1) amino acid differences per framework region. Optionally a variant antibody has at most 2 (optionally at most 1) amino acid differences total in the framework regions thereof when compared to a corresponding reference (anifrolumab) antibody, with the proviso that there is at most 2 amino acid differences per framework region. Optionally a variant antibody has at most 2 (optionally at most 1) amino acid differences total in the framework regions thereof when compared to a corresponding reference (anifrolumab) antibody, with the proviso that there is at most 1 amino acid difference per framework region.

A variant antibody may comprise a variable heavy chain and a variable light chain as described herein, wherein: the heavy chain has at most 14 amino acid differences (at most 2 amino acid differences in each CDR and at most 2 amino acid differences in each framework region) when compared to a heavy chain sequence herein; and the light chain has at most 14 amino acid differences (at most 2 amino acid differences in each CDR and at most 2 amino acid differences in each framework region) when compared to a light chain sequence herein; wherein the variant antibody binds to the same target antigen as the reference (anifrolumab) antibody (e.g. IFNAR) and preferably with the same affinity.

The variant heavy or light chains may be referred to as “functional equivalents” of the reference heavy or light chains. A variant antibody may comprise a variable heavy chain and a variable light chain as described herein, wherein: the heavy chain has at most 7 amino acid differences (at most 1 amino acid difference in each CDR and at most 1 amino acid difference in each framework region) when compared to a heavy chain sequence herein; and the light chain has at most 7 amino acid differences (at most 1 amino acid difference in each CDR and at most 1 amino acid difference in each framework region) when compared to a light chain sequence herein; wherein the variant antibody binds to the same target antigen as the reference (anifrolumab) antibody (e.g. IFNAR) and optionally with the same affinity.

### Functional Variants of Anifrolumab

Functional variants of anifrolumab include the antibodies described in WO 2018/023976 A1, incorporated herein by reference (Table 2).

TABLE 2
anti-IFNAR antibody sequences

| | | | | |
|---|---|---|---|---|
| H15D10 | 19 | VH | EVQLVQSGAE VKKPGESLRISC | SEQ ID |
| | | | KGSGYTFT NYWVAWVRQM | NO: 13 |
| | | | PGKGLESMGI IYPGDSDTRY | |
| | | | SPSFQGHVTI SADKSISTAY | |
| L8C3 | 21 | VL | DIQMTQSPSSLSASLGDRVTIT | SEQ ID |
| | | | CRASQNVGNYLNWYQQKPGKAP | NO: 14 |
| | | | KLLIYRASNLASGVPSRFSGSG | |
| | | | SGTDFTLTISSLQPEDFATYYC | |
| | | | QQMEHAPPTFGQGTKVEIKR | |
| L16C11 | 22 | VL | EIVLTQSPGTLSLSPGERATLS | SEQ ID |
| | | | CRASQSVIGYYLAWYQQKPGQA | NO: 15 |
| | | | PRLLIYSVSTLASGIPDRFSGS | |
| | | | GSGTDFTLTISRLEPEDFAVYY | |
| | | | CQQYYRFPITFGQGTKVEIK | |
| H19B7 | 20 | VH | EVQLVQSGAE VKKPGESLRI | SEQ ID |
| | | | SCKGSGYTFT NYWMAWVRQM | NO: 16 |
| | | | PGKGLESMGI IYPSDSDTRY | |
| | | | SPSFQGHVTI SADKSISTAY | |
| | | | LOWSSLKASD TAMYYCARHD | |
| | | | VEGYDYWGQG TLVTVSS | |

Functional variants include antibodies comprising the VH amino acid sequence SEQ ID NO: 13. Functional variants include antibodies comprising the VH amino acid sequence SEQ ID NO: 16. Functional variants include antibodies comprising the VL amino acid sequence SEQ ID NO: 14. Functional variants include antibodies comprising the VL amino acid sequence SEQ ID NO: 15. Functional variants include antibodies comprising the VL amino acid sequence SEQ ID NO: 16. Functional variants include antibodies comprising the VH sequence SEQ ID NO: 13 and VL amino acid sequence SEQ ID NO: 16. Functional variants include antibodies comprising the VH sequence SEQ ID NO: 13 and VL amino acid sequence SEQ ID NO: 15. Functional variants include antibodies comprising the VH sequence SEQ ID NO: 16 and VL amino acid sequence SEQ ID NO: 15. Functional variants include antibodies comprising the VH sequence SEQ ID NO: 16 and VL amino acid sequence SEQ ID NO: 14.

IFNAR inhibitors may be a monoclonal antibody comprising the VH amino acid sequence SEQ ID NO: 13. The anti-IFNAR antibodies may comprise the VH amino acid sequence SEQ ID NO: 16. The anti-IFNAR antibodies may comprise the VL amino acid sequence SEQ ID NO: 14. The anti-IFNAR antibodies may comprise the VL amino acid sequence SEQ ID NO: 15. The anti-IFNAR antibodies may comprise the VL amino acid sequence SEQ ID NO: 16. The anti-IFNAR antibodies may comprise the VH sequence SEQ ID NO: 13 and VL amino acid sequence SEQ ID NO: 16. The anti-IFNAR antibodies may comprise the VH sequence SEQ ID NO: 13 and VL amino acid sequence SEQ ID NO: 15. The anti-IFNAR antibodies may comprise the VH sequence SEQ ID NO: 16 and VL amino acid sequence SEQ ID NO: 15. The anti-IFNAR antibodies may comprise the VH sequence SEQ ID NO: 16 and VL amino acid sequence SEQ ID NO: 14.

### QX006N

Functional variants of anifrolumab and anti-IFNAR antibodies include the QX006N antibody described in CN 11327807, incorporated herein by reference.

TABLE 3
QX006N antibody sequences

| | SEQ | |
|---|---|---|
| | ID | |
| Description | NO | Sequence |
| QX006N (VH) | 17 | EVQLVESGGGLVQPGGSLRLSCAAS |
| | | GFSLSSYYMTWVRQAPGKGLEWVSV |
| | | INVYGGTYYASWAKGRFTISRDNSK |
| | | NTLYLQMNSLRAEDTAVYYCAREDV |
| | | AVYMAIDLWGQGTLVTVSS |
| QX006N (VL) | 18 | AIQMTQSPSSLSASVGDRVTITCQA |
| | | SQSISNQLSWYQQKPGKAPKLLIYD |
| | | ASSLASGVPSRFSGSRSGTKFTLTI |
| | | SSLQPEDFATYYCLGIYGDGADDGI |
| | | AFGGGTKVEIK |
| QX006N (HCDR1) | 19 | SYYMT |
| QX006N (HCDR2) | 20 | VINVYGGTYYASWAKG |
| QX006N (HCDR3) | 21 | EDVAVYMAIDL |
| QX006N (LCDR1) | 22 | QASQSISNQLS |
| QX006N (LCDR2) | 23 | DASSLAS |
| QX006N (LCDR3) | 24 | LGIYGDGADDGIA |

IFNAR inhibitors may be a monoclonal antibody comprising the VH amino acid sequence SEQ ID NO: 17. The anti-IFNAR antibodies may comprise the VL amino acid sequence SEQ ID NO: 18.

QX006N is an immunoglobulin comprising an HCDR1, HCDR2 and HCDR3 of SEQ ID NO: 19, SEQ ID NO: 20, and SEQ ID NO: 21, respectively (or functional variant thereof); and an LCDR1, LCDR2 and LCDR3 of SEQ ID NO: 22, SEQ ID NO: 23, and SEQ ID NO: 23, respectively (or functional variant thereof). QX006N is an immunoglobulin comprising a VH amino acid sequence SEQ ID NO: 17 the VL amino acid sequence SEQ ID NO: 18.

### Anifrolumab in the Clinic

Anifrolumab safety has been evaluated in 8 blinded or open-label intravenous (IV) and subcutaneous (SC) studies: 6 studies in patients with SLE (Study 05, Study 04, Study 1013, Study 1145, and Study 08), 1 study in patients with systemic sclerosis (SSc) (Study MI-2P980), and 1 study in healthy volunteers (Study 06) (Table 4). Of these studies, two (Studies 08 and 06) employed SC anifrolumab administration. Two studies are ongoing: 1 study in patients with SLE (Study 09) and 1 study in patients with lupus nephritis (LN) (Study 07).

TABLE 4
Anifrolumab clinical studies

| | | | |
|---|---|---|---|
| Subjects | Admin. | Anifro Dose | CT. gov |

Phase III studies

| | | | | | |
|---|---|---|---|---|---|
| Study 05 | TULIP II | SLE patients | IV | 300 mg Q4W | NCT02446899 |
| Study 04 | TULIP I | SLE patients | IV | 300 mg Q4W | NCT02962960 |
| Study 09 | Long-term | SLE patients | IV | 300 mg | |
| | extension | | | | |

Phase II studies

| | | | | | |
|---|---|---|---|---|---|
| Study 1013 | MUSE | SLE patients | IV | 300 mg or | NCT01438489 |
| | | | | 1000 mg | |
| | | | | Q4W | |
| Study 1145 | MUSE OLE | | IV | 300 mg | NCT01753193 |
| Study 08 | | SLE patients | SC | | NCT02962960 |
| Study 07 | | LN patients | | | NCT02547922 |

Phase I

| | | | | |
|---|---|---|---|---|
| Study MI-CP180 | Scleroderma patients | IV | | |
| Study 06 [12] | Healthy volunteers | IV and SC | 300 mg, SC, | NCT02601625 |
| | | | 300 mg IV or | |
| | | | 600 mg SC | |

Study 1013 is described in further detail in Furie et al. 2017 [13], which is incorporated herein by reference in its entirety. Study 04 is described in further detail in Furie et al. 2019 [14], which is incorporated herein by reference in its entirety. The results of Study 05 are presented in Morand et al. 2020 [15], herein incorporated by reference in its entirety. A full summary of the evidence for intravenous anifrolumab clinical efficacy in SLE is provided in Tanaka et al., 2020[16], which is incorporated herein by reference in its entirety.

### Formulations

Stable formulations suitable for administration to subjects and comprising anifrolumab are described in detail in U.S. patent Ser. No. 10/125,195 B1, which is incorporated herein in its in entirety.

The Examples that follow are illustrative of specific embodiments of the disclosure, and various uses thereof. They are set forth for explanatory purposes only and should not be construed as limiting the scope of the disclosure in any way.

### Type I IFN Gene Signature (IFNGS)

Type I IFN is considered to play a central role SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of type I IFN remains a challenge. As such, a transcript-based marker was developed to evaluate the effect of over expression of the target protein on a specific set of mRNA markers. The expression of these markers is easily detected in whole blood and demonstrates a correlation with expression in diseased tissue such as skin in SLE. The bimodal distribution of the transcript scores for SLE subjects supports defining an IFN test high and low subpopulation (Error! Reference source not found.). The type I IFN test is described in WO2011028933 A1, which is incorporated herein by reference in its entirety. The type I IFN gene signature may be used to identify a subject has a type I IFN gene signature (IFNGS)-test high patient or an IFNGS-test low patient. The IFNGS test measures expression of the genes IFI27, IFI44, IFI44L, and RSAD2 compared with 3 reference genes; 18S, ACTB and GAPDH in the whole blood of the subject. The result of the test is a score that is compared with a pre-established cut-off that classifies patients into 2 groups with low or high levels of IFN inducible gene expression (Error! Reference source not found.).

The expression of the genes may be measured by RT-PCR. Suitable primers and probes for detection of the genes may be found in WO2011028933. A suitable kit for measuring gene expression for the IFNGS test is the QIAGEN Therascreen® IFIGx RGQ RT-PCR kit (IFIGx kit), as described in Brohawn et al. [17], which is incorporated herein by reference in its entirety.

### COVID-19 Vaccines

Example vaccines for COVID-19 are shown in Table 5 below.

TABLE 5
COVID-19 vaccines

| Vaccine | Vaccine | Type of | | | | |
|---|---|---|---|---|---|---|
| platform | platform | candidate | Number | | Route of | |
| acronym | description | vaccine | of doses | Schedule | administration | Developers |
| IV | Inactivated | CoronaVac; | 2 | Day 0 + 14 | IM | Sinovac |
| | virus | inactivated | | | | Research and |
| | | SARS-CoV-2 | | | | Development |
| | | vaccine (vero | | | | Co., Ltd |
| | | cell) | | | | |
| IV | Inactivated | Inactivated | 2 | Day 0 + 21 | IM | Sinopharm; |
| | virus | SARS-CoV-2 | | | | |
| | | vaccine (Vero | | | | |
| | | cell) | | | | |
| IV | Inactivated | Inactivated | 2 | Day 0 + 21 | IM | Sinopharm; |
| | virus | SARS-CoV-2 | | | | |
| | | vaccine (Vero | | | | |
| | | cell), vaccine | | | | |
| | | name BBIBP- | | | | |
| | | CorV | | | | |
| VVnr | Viral vector | ChAdOx1-S - | 1-2 | Day 0 + 28 | IM | AstraZeneca + |
| | (Non- | (AZD1222) | | | | University of |
| | replicating) | | | | | Oxford |
| VVnr | Viral vector | Recombinant | 1 | Day 0 | IM | CanSino |
| | (Non- | novel | | | | Biological |
| | replicating) | coronavirus | | | | Inc./Beijing |
| | | vaccine | | | | Institute of |
| | | (Adenovirus | | | | Biotechnology |
| | | type 5 vector) | | | | |
| VVnr | Viral vector | Recombinant | 1 | Day 0 | IH | CanSino |
| | (Non- | COVID-19 | | | | Biological |
| | replicating) | vaccine | | | | Inc./Beijing |
| | | (adenovirus | | | | Institute of |
| | | type 5 vector) | | | | Biotechnology |
| | | for Inhalation | | | | |
| | | (Ad5-nCoV-IH) | | | | |
| VVnr | Viral vector | Ad26.COV2.S | 1-2 | Day 0 or | IM | Janssen |
| | (Non- | | | Day 0 + 56 | | Pharmaceutical |
| | replicating) | | | | | |
| RNA | RNA based | mRNA-1273 | 2 | Day 0 + 28 | IM | Moderna + |
| | vaccine | (Spikevax) | | | | National Institute |
| | | | | | | of Allergy and |
| | | | | | | Infectious |
| | | | | | | Diseases (NIAID) |
| RNA | RNA based | BNT162b2 (3 | 2 | Day 0 + 21 | IM | Pfizer/BioNTech + |
| | vaccine | LNP-mRNAs), | | | | Fosun Pharma |
| | | also known as | | | | |
| | | “Comirnaty” | | | | |
| PS | Protein | MVC- | 2 | Day 0 + 28 | IM | Medigen Vaccine |
| | subunit | COV1901 | | | | Biologics + |
| | | (Spike-2P | | | | Dynavax + |
| | | protein + | | | | National Institute |
| | | adjuvant CpG | | | | of Allergy and |
| | | 1018) | | | | |
| RNA | RNA based | mRNA- | 3 | Day 0 or | IM | Moderna + |
| | vaccine | 1273.351. | | Day 0 + 28 | | National Institute |
| | | | | | | of Allergy and |
| | | | | | | Infectious |
| | | | | | | Diseases (NIAID) |

SARS-CoV-2 vaccines include for following: intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (InCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), SARS-Cov-2 coronavirus vaccine NVX-CoV2373 (Novavax), an intramuscular vaccine INO-4700 (GLS-5300) (Inovio Pharma and GeneOne Life Science), and combinations thereof.

### Example 1: a Randomized, Placebo-Controlled Phase 3 Extension Trial of the Long-Term Safety and Tolerability of Anifrolumab in Active Systemic Lupus Erythematosus

### Summary

### Objective

To explore long-term safety and tolerability of anifrolumab 300 mg vs placebo in patients who completed a TULIP trial and enrolled in the placebo-controlled 3-year extension study (CT.gov reference NCT02794285).

### Methods

Patients received anifrolumab 300 mg or placebo every 4 weeks in the blinded extension. Primary comparisons in the long-term extension (LTE) were between patients who received anifrolumab 300 mg or placebo throughout both a TULIP study and the LTE. For rare safety events, comparisons also included patients who received any dose of anifrolumab during a TULIP study or the LTE. When exposure differed, rates were adjusted for exposure (EAIR) per 100 patient-years.

### Results

In the LTE period, rates of serious adverse events (SAEs) were numerically lower with anifrolumab vs placebo (8.5 vs 11.2), including AEs leading to discontinuation of investigational product (2.5 vs 3.2). Rates of non-opportunistic serious infections were comparable between groups (3.7 vs 3.6). Rates of COVID-related AEs, including asymptomatic infections, were higher with anifrolumab vs placebo (10.2 vs 6.3). Surprisingly, no COVID-related AEs, including SAEs, occurred in fully vaccinated individuals. Rates of malignancy and major acute cardiovascular events were low and comparable between anifrolumab and placebo. Treatment with anifrolumab was associated with lower cumulative glucocorticoid use and greater mean improvement in SLEDAI-2K compared with placebo.

### Conclusion

This LTE study represents the longest placebo-controlled clinical trial performed in SLE to date. No new safety findings were identified in the LTE, supporting the favorable benefit-risk profile of anifrolumab for patients with moderate to severe SLE receiving standard therapy. No COVID AEs occurred in patients after they were fully vaccinated.

### Introduction

Reported here are results of the first long-term placebo-controlled trial in SLE to characterize the safety and tolerability of intravenous anifrolumab versus placebo in patients who had moderate to severe SLE despite standard therapy. To be eligible for this placebo-controlled long-term extension (LTE) study (NCT02794285), patients had to complete a phase 3 TULIP trial (NCT02446912 or NCT02446899) through the 52-week double-blind treatment period. Given the circumstances of the COVID-19 pandemic emerging in the final year of this global multicenter extension study, this is also the first report on the safety profile of an investigational biologic in SLE during the pre- and post-vaccination periods of the COVID-19 pandemic.

### Methods

### Study Design

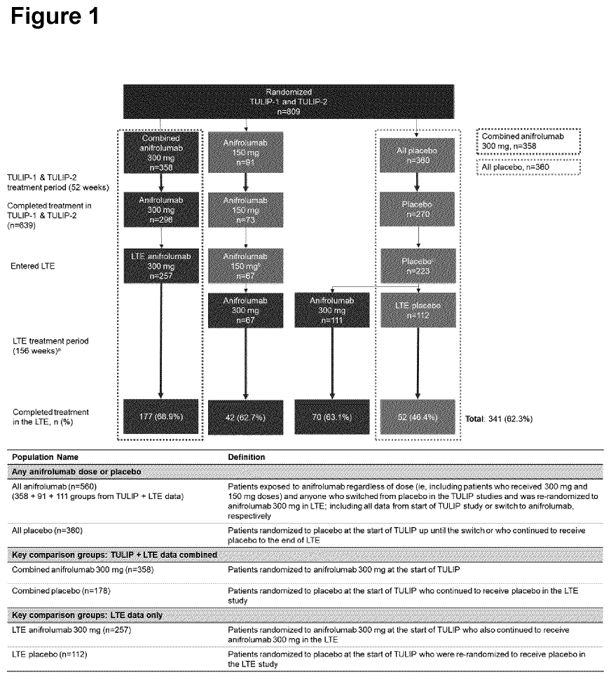

This study reports on a 3-year, phase 3, randomized, double-blind, placebo-controlled, LTE study (15) conducted across 176 study sites in 24 countries in patients who completed the 52 week double-blind treatment period of one of the phase 3 TULIP trials (TULIP-1: NCT02446912 or TULIP-2: NCT02446899) [19,20]. Patients were required to have moderate to severe SLE at the time of TULIP-1 or TULIP-2 randomization, and were allowed to participate in the extension study upon re-consent; the start of the LTE was the end of the double-blind treatment period of the TULIP trials (FIG. 1). At extension study enrollment, patients previously treated with anifrolumab 300 mg remained on blinded anifrolumab 300 mg; patients previously treated with anifrolumab 150 mg in TULIP-1 were switched to blinded anifrolumab 300 mg; and patients previously randomized to placebo were re-randomized 1:1 to blinded anifrolumab 300 mg or placebo by an Interactive Voice/Web Response System algorithm to give an approximate final anifrolumab 300 mg to placebo ratio of 4:1.

For primary safety, the main comparison groups were between patients during the 3-year LTE study who received anifrolumab 300 mg in both TULIP and the LTE (“LTE anifrolumab 300 mg”) versus those who received placebo for the same time frame (“LTE placebo,” FIG. 1). For rare safety events (eg, malignancy, major acute cardiovascular events), data throughout the 4-year TULIP and LTE period were used, and the main comparison groups were patients with any anifrolumab exposure (“all anifrolumab” group) versus patients with any placebo exposure (“all placebo” group; FIG. 1). Data from patients randomized to placebo in TULIP and then anifrolumab 300 mg in the LTE are included in the “all anifrolumab” group after the first dose of anifrolumab in the LTE. As anifrolumab 150 mg is not the recommended dose for SLE, patients who switched from anifrolumab 150 mg in TULIP-1 to the 300-mg dose in the LTE were included in the all anifrolumab group and will only be described in the context of this group for the purposes of this analysis.

For efficacy comparisons, including disease activity and steroid use, the main comparison groups during the 4-year TULIP and LTE period were patients who received anifrolumab 300 mg or placebo in the TULIP studies and continued on their same treatments throughout the LTE (“combined anifrolumab 300 mg” versus “combined placebo” groups, respectively). Note, the key difference between these groups and the LTE anifrolumab 300 mg and LTE placebo groups was the time frame analyzed (TULIP and LTE: Years 1-4 compared with LTE only: Years 2-4).

Patients received intravenous anifrolumab 300 mg or placebo every 4 weeks after the start of the LTE (following the last visit in the TULIP study) for a total of up to 39 doses. After the 156-week treatment period in the LTE (last dose of investigational product given at Week 152), patients continued in the study for another 8 weeks to complete a 12-week safety follow-up period after their last dose. This study was conducted in accordance with principles of the Declaration of Helsinki and the International Conference on Harmonisation Guidance for Good Clinical Practice. All patients provided informed consent and the study was approved by the ethics committee or institutional review board.

### Patients

Patients enrolled in the LTE must have completed the 52-week treatment in either TULIP-1 or TULIP-2. Full details of inclusion and exclusion criteria for the TULIP trials have been previously described [19,20] and in brief below. The extension study was designed to reflect real-world clinical practice, allowing investigators to add or change background standard of care treatment, including immunosuppressants and glucocorticoids, based on clinical judgment. However, use of cyclophosphamide, other biologics, intravenous immunoglobulin, or intravenous glucocorticoids was not permitted.

### TULIP Studies

For inclusion criteria in the TULIP studies, briefly, patients were aged 18-70 years and were required to have a Systemic Lupus Erythematosus Disease Activity Index 2000 (SLEDAI-2K) score of ≥6; clinical SLEDAI-2K score of ≥4; British Isles Lupus Assessment Group 2004 (BILAG-2004) organ domain score of ≥1 A item or 2 B items; Physician's Global Assessment (PGA) score of ≥1 (0-3 scale); seropositive for antinuclear antibodies, anti-double-stranded DNA (anti-dsDNA), or anti-Smith antibodies; and ongoing stable treatment with at least one of either prednisone or equivalent, an antimalarial, azathioprine, mizoribine, mycophenolate mofetil, mycophenolic acid, or methotrexate.

### LTE Study

For inclusion in the LTE study, patients had to have received anifrolumab or placebo in one of the TULIP trials and completed the 52-week double-blind treatment period, and had to provide written informed consent before participating in any protocol-related procedures. Patients were excluded if they had received any condition that, in the opinion of the investigator, would interfere with evaluation of the investigational product or interpretation of patient safety or study results; patients were excluded if they had concurrent enrollment in another clinical study that was not TULIP-1 or TULIP-2. For exclusion criteria related to concomitant medications, patients were also excluded if they received any of the following within the last 60 days: azathioprine >200 mg/day; mycophenolate mofetil >2.0 g/day; mycophenolic acid >1.44 g/day; oral, subcutaneous, or intramuscular methotrexate >25 mg/week; or mizoribine >150 mg/day.

### Safety Assessments

AE data were recorded throughout the entire study and were categorized as during treatment or during study according to the onset date based on administration of the last dose, whether in the TULIP trials or the extension study. AEs that occurred during treatment were defined as an AE with onset from the day of first dose of study treatment through the day of last dose of study treatment plus 28 days or end of study date, whichever was earliest. AEs that occurred during the study are defined as an AE with onset from the day of first dose of study treatment through to the end of study day.

### Tuberculosis Screening and Monitoring During the LTE Study

If a patient had a newly positive QFT-G test result for tuberculosis at the time of randomization in the LTE study, they started prophylaxis within 30 days of randomization, but before the second dose of investigational product in the LTE. Each patient was monitored at every visit in the LTE to ensure that no signs or symptoms of active tuberculosis were present, no recent contact with anyone with active tuberculosis, and there is no history of latent or active tuberculosis. Patients identified with latent tuberculosis were assessed by a local tuberculosis specialist to confirm the diagnosis and local standard of care that was used in treatment. Once latent tuberculosis was confirmed, treatment was started immediately and no investigational product was administered until treatment of latent tuberculosis had begun. Additionally, patients with newly diagnosed latent tuberculosis must have agreed to complete a locally recommended course of treatment for latent tuberculosis to continue to receive investigational product.

### Efficacy Assessments: SLEDAI-2K, PGA, Glucocorticoids, Flares and SDI

SLEDAI-2K was reported for Weeks 0 (TULIP baseline), 24, 52, 64, 76, 88, 104, 128, 156, 180, and 208, as well as at Weeks 212 and 216 post-final dose during follow-up. PGA was reported for Weeks 0, 24, 52, 64, 76, 88, 104, 128, 156, 180, and 208. Glucocorticoid dosage in the study was collected at each visit and is reported here by year (baseline and to Year 4) during the TULIP and extension periods. Systemic Lupus International Collaborating Clinics/American College Rheumatology Damage Index (SDI) global score was collected at Weeks 52, 104, 156, and 208. Glucocorticoid dosage in the study was collected at each visit and is reported here by year (baseline and to Year 4) during the TULIP and extension periods.

Flares were assessed using the modified Safety of Estrogens in Lupus Erythematosus National Assessment (SELENA) Flare Index, which includes SLEDAI-2K. A mild to moderate flare was defined as at least one of an increase in SLEDAI-2K of ≥3 points but <7 points from the previous visit; at least one new or worse manifestation in discoid, photosensitivity, profundus, cutaneous vasculitis, bullous lupus, nasopharyngeal ulcers, pleuritic, pericarditis, arthritis, or SLE fever; or an increase in PGA of ≥1 point from the previous visit but a PGA value of ≤2.5 points. A severe flare was defined as at least one of an increase in SLEDAI-2K of ≥7 points from the previous visit; at least one new or worse manifestation in central nervous system SLE, vasculitis, nephritis, myositis, or hemolytic anemia; hospitalization due to SLE disease activity; or an increase in PGA to >2.5 points.

There was no imputation for missing data for patients who discontinued or were lost to follow-up, with the exception of missing SDI global score. In this case, if only one of “proteinuria ≥3.5 g/day” or “end-stage renal disease (irrespective of dialysis or transplantation)” was missing, the score was computed. SDI global score was set to missing if both items described were absent or any other item was missing. Missing SDI scores were imputed based on worst observation, for both missing intermediate values and decreasing scores. Because the SDI global score should never decrease, an item level reduction is replaced by carrying the worst (highest) observation forward (WOCF). WOCF will be applied to all items, including intermittent missing values (prior to discontinuation of investigational product). Time to first SDI worsening is defined as the date of first SDI worsening minus date of first administration of investigational product. If the patient did not have a worsening, the time to SDI worsening will be censored at the end of the exposure time, or Week 208, whichever occurs earlier.

### Statistical Analyses

Baseline demographics and characteristics are presented as descriptive statistics by treatment groups. AEs are summarized by descriptive statistics, including exposure-adjusted incidence rates (EAIRs) and adjusted cumulative proportions. Exposure was calculated up until the earlier of either the date of last dose of treatment+84 days or the date of study discontinuation, death, or withdrawal of consent. COVID-related events are described by event rates based on time at risk during the pandemic.

Observed values and changes from baseline in SLEDAI-2K, SDI, and PGA are presented by visit with descriptive statistics. Mean change from baseline in SLEDAI-2K is analyzed using analysis of covariance including baseline value (continuous), treatment group, visit, and randomization stratification factors reduced to factors type I IFN gene signature test result at screening and glucocorticoid dose at baseline. For glucocorticoid use, standardized area under the curve (AUC) and the proportion of patients by glucocorticoid dose are both presented by treatment group and by year with summary statistics. Sensitivity analyses for glucocorticoid use were conducted to exclude patients who had a glucocorticoid dose >40 mg/day at TULIP study baseline due to unknown end dates. Four patients that had missing end dates for glucocorticoids starting before randomization were excluded, as these high dosages were carried throughout the studies, inflating the results. Flare rate per patient year is presented for mild to moderate flares, severe flares, and overall.

### Definitions of EAIR and Event Rate

The EAIR per 100 patient-years is defined as the number of patients with the specific event divided by the total exposure in years ×100. The exposure time is defined as from the date of first administration of treatment to death, end of treatment plus 84 days, or end of study, whichever comes first. The event rate per 100 patient-years is defined as the number of patients with an event divided by the total time at risk during the pandemic in years ×100. Time at risk is defined as from the date of start of the pandemic to death, end of treatment plus 84 days, or end of study, whichever comes first. When reporting events occurring during treatment only, end of period plus 28 days instead of 84 days is considered.

### Study Outcomes

The primary outcome was long-term safety and tolerability as assessed by the rates of adverse events (AEs), serious adverse events (SAEs) including those leading to deaths, adverse events leading to treatment discontinuation (DAE), and adverse events of special interest (AESIs). Safety information was collected at every visit. Exploratory efficacy outcomes included Systemic Lupus Erythematosus Disease Activity Index 2000 (SLEDAI-2K), Physician's Global Assessment (PGA), glucocorticoid use, flare incidence and severity, and Systemic Lupus International Collaborating Clinics/American College Rheumatology Damage Index (SDI) global score. Further information on study outcomes is described in Supplemental Methods.

### COVID-19 Pandemic

The LTE started on Jun. 30, 2016 and continued during the COVID-19 pandemic, which was declared on Mar. 11, 2020 by the World Health Organization. Total exposure during the pandemic was calculated for each patient as:

Total exposure=end of period −start date of the pandemic+1

Patients were considered to be fully vaccinated against COVID-19 if they received at least 2 doses of any approved vaccine, as was the standard COVID-19 vaccination schedule in the relevant time period.

### Results

### Patients

Out of 639 patients who completed treatment in the TULIP studies, 547 enrolled in the LTE and were randomized to receive at least one dose of treatment (FIG. 1). There were 257 patients who continued anifrolumab 300 mg (LTE anifrolumab 300 mg), and 67 patients transitioned from anifrolumab 150 mg to 300 mg. The 223 patients who received placebo in TULIP-1 or TULIP-2 were re-randomized 1:1 to anifrolumab 300 mg (n=111) or placebo (n=112, LTE placebo) in the LTE. Among patients who would go on to comprise the primary safety comparison groups in the LTE study period, previously described as LTE anifrolumab 300 mg (n=257) and LTE placebo (n=112) (FIG. 1), demographics and baseline disease characteristics at the start of the TULIP studies were generally well balanced between groups (Table 6), and similar proportions of patients were receiving glucocorticoids or immunosuppressants. For patients who continued on to the LTE, disease activity scores at TULIP baseline, as measured by mean (SD) SLEDAI-2K global scores, were 11.2 (3.7) in the LTE anifrolumab 300 mg group and 11.3 (3.6) in the LTE placebo group. This was consistent with the overall TULIP population.

TABLE 6
Demographics and SLE disease characteristics at TULIP baseline
for patients who continued treatment in the LTE study

| | LTE Anifrolumab 300 mg | | LTE Placebo | |
|---|---|---|---|---|
| Characteristic | (n = 257) | | (n = 112) | |
| Age, mean (SD), years | 43.4 | (12.0) | 41.4 | (11.5) |
| Sex, female, n (%) | 237 | (92.2) | 103 | (92.0) |
| Race, n (%) | | | | |
| White | 173 | (67.3) | 77 | (68.8) |
| Black | 28 | (10.9) | 11 | (9.8) |
| Asian | 33 | (12.8) | 10 | (8.9) |
| Other | 15 | (5.8) | 11 | (9.8) |
| Ethnicity, Hispanic or Latino, n (%) | 54 | (21.0) | 28 | (25.0) |
| Geographic region, n (%) | | | | |
| USA/Canada | 98 | (38.1) | 44 | (39.3) |
| Europe | 90 | (35.0) | 41 | (36.6) |
| Latin America | 33 | (12.8) | 15 | (13.4) |
| Asia Pacific | 31 | (12.1) | 8 | (7.1) |
| Other (rest of world) | 5 | (1.9) | 4 | (3.6) |
| Time from initial SLE diagnosis to randomization, months | | | | |
| Median (range) | 92.0 | (0-555) | 80.5 | (6-503) |
| SLEDAI-2K | | | | |
| Mean (SD) | 11.2 | (3.7) | 11.3 | (3.6) |
| Score ≥10, n (%) | 184 | (71.6) | 80 | (71.4) |
| PGA score, mean (SD) | 1.8 | (0.4) | 1.8 | (0.4) |
| SDI global score, mean (SD) | 0.6 | (1.1) | 0.6 | (0.9) |
| Type I IFNGS high, n (%) | 206 | (80.2) | 93 | (83.0) |
| ANA positive, n (%) | 229 | (89.1) | 99 | (88.4) |
| Anti-dsDNA positive, n (%) | 113 | (44.0) | 38 | (33.9) |
| Abnormal (low) complement C3, n (%) | 90 | (35.0) | 36 | (32.1) |
| Abnormal (low) complement C4, n (%) | 56 | (21.8) | 19 | (17.0) |
| Baseline SLE treatments | | | | |
| Glucocorticoids (prednisone or equivalent), n (%) | 208 | (80.9) | 92 | (82.1) |
| Antimalarials, n (%) | 171 | (66.5) | 83 | (74.1) |
| Azathioprine, n (%) | 42 | (16.3) | 18 | (16.1) |
| Methotrexate, n (%) | 45 | (17.5) | 26 | (23.2) |
| Mycophenolate, n (%) | 37 | (14.4) | 14 | (12.5) |
| NSAIDs, n (%) | 27 | (10.5) | 14 | (12.5) |

anti-dsDNA, anti-double-stranded DNA; ANA, antinuclear antibody; BILAG-2004, British Isles Lupus Assessment Group 2004; C3, complement 3; C4, complement 4; IFNGS, interferon gene signature; LTE, long-term extension; NSAIDs, non-steroidal anti-inflammatory drugs; PGA, Physician's Global Assessment; SD, standard deviation; SDI, Systemic Lupus International Collaborating Clinics/American College of Rheumatology Damage Index; SLE, systemic lupus erythematosus; SLEDAI-2K, SLE Disease Activity Index 2000.

Of the patients in the LTE anifrolumab 300 mg group, 69.3% (178/257) completed the 3-year extension study, compared with 48.2% (54/112) of the patients in the LTE placebo group. The most commonly reported reasons for discontinuation were withdrawal by patient and lack of efficacy, with higher percentages observed in the LTE placebo group compared with the LTE anifrolumab 300 mg group. Eight patients discontinued treatment because of the COVID pandemic (LTE anifrolumab 300 mg group: n=7 [2.7%]; LTE placebo group: n=1 [0.9%]).

Overall exposure to anifrolumab during the extension study in the LTE anifrolumab 300 mg group was 683.5 patient years compared with 250.3 patient years of exposure to placebo in the LTE placebo group (Table 7). The total exposure to anifrolumab across all doses and all groups in the TULIP studies and the 3 years of the LTE was 1,568 patient years.

TABLE 7
AEs, deaths, and exposure-adjusted incidence rates in any category
during treatment and follow-up in the extension study

| | LTE Anifrolumab 300 mg | | | LTE Placebo | | |
|---|---|---|---|---|---|---|
| | (n = 257) | | | (n = 112) | | |
| | Exposure^{a} | | | | | |
| | 683.5 years | | | 250.3 years | | |
| | | | EAIR^{b} (per | | | EAIR^{b} (per |
| | n (%) | | 100 PY) | n (%) | | 100 PY) |
| Any AE | 266 | (87.9) | 33.1 | 94 | (83.9) | 37.6 |
| Any AE with outcome of death | 3 | (1.2) | 0.4 | 1 | (0.9) | 0.4 |
| Any AE leading to treatment | 17 | (6.6) | 2.5 | 8 | (7.1) | 3.2 |
| discontinuation | | | | | | |
| Any AE of severe intensity | 43 | (16.7) | 6.3 | 13 | (11.6) | 5.2 |
| Any SAE (including events with outcome | 58 | (22.6) | 8.5 | 28 | (25.0) | 11.2 |
| of death) | | | | | | |
| Any AESI | 75 | (29.2) | 11.0 | 24 | (21.4) | 9.6 |
| Any AESI of non-opportunistic serious | 25 | (9.7) | 3.7 | 9 | (8.0) | 3.6 |
| infections | | | | | | |
| Any AESI of herpes zoster | 23 | (8.9) | 3.4 | 7 | (6.3) | 2.8 |
| Any AESI of latent tuberculosis^{c} | 16 | (6.2) | 2.3 | 2 | (1.8) | 0.8 |
| Any AESI of influenza | 15 | (5.8) | 2.2 | 2 | (1.8) | 0.8 |
| Any AESI of major adverse cardiovascular | 5 | (1.9) | 0.7 | 3 | (2.7) | 1.2 |
| events^{d} | | | | | | |
| Any AESI of malignancy | 2 | (0.8) | 0.3 | 2 | (1.8) | 0.8 |

| Any AESI of anaphylaxis | 0 | 0.0 | 0 | | 0.0 |
|---|---|---|---|---|---|
| Any AESI of opportunistic infections | 0 | 0.0 | 3 | (2.7) | 1.2 |

| | | | | |
|---|---|---|---|---|
| Any AESI of vasculitis | 0 | 0.0 | 0 | 0.0 |

AE, adverse event; AESI, adverse event of special interest; CI, confidence interval; EAIR, exposure-adjusted incidence rate; IFN, interferon; LTE, long-term extension; PY, patient-years; SAE, serious adverse event; SLE, systemic lupus erythematosus.
^{a}Exposure in days for each patient was calculated as the earlier of either (date of last dose of treatment + 84 days, or date of study discontinuation) − date of first dose of treatment + 1 day.
^{b}The EAIR per 100 patient-years is defined as the number of patients with the specific event divided by the total exposure in years × 100. The exposure time is defined as from the date of first administration of treatment to death, end of treatment plus 84 days, or end of study, whatever comes first.
^{c}Latent tuberculosis was defined as a positive IFN-gamma-release assay. No active cases of tuberculosis were reported.
^{d}According to the cardiovascular event adjudication committee.

### Safety and Tolerability

The safety profile of anifrolumab in SLE during the 1-year TULIP studies has been published (5, 6). Focusing on a comparison of the LTE anifrolumab 300 mg group vs the LTE placebo group, exposure-adjusted incidence rates (EAIRs) of any AE (33.1 vs 37.6), any SAE including events with the outcome of death (8.5 vs 11.2), and any DAE (2.5 vs 3.2) were all lower in the LTE anifrolumab 300 mg group compared with the LTE placebo group, respectively (Table 7). The safety profile of anifrolumab generated among those patients who received any dose of anifrolumab (all anifrolumab group; n=560), whether in one of the TULIP studies or the LTE, was similar to the profile generated in the combined TULIP and LTE dataset (combined anifrolumab 300 mg group; n=358) by those patients who received anifrolumab 300 mg (Table 8).

TABLE 8
AEs, SAEs, deaths, AESIs, and exposure-adjusted incidence rates in
any category during treatment and follow-up through Weeks 0-216^{a}

| | All Anifrolumab | | | Combined Anifrolumab | | | All Placebo | | |
|---|---|---|---|---|---|---|---|---|---|
| | (n = 560) | | | 300 mg (n = 358) | | | (n = 360) | | |
| | Exposure^{b} | | | | | | | | |
| | 1568.0 years | | | 1026.2 years | | | 587.1 years | | |
| | | | EAIR^{c} | | | EAIR^{c} | | | EAIR^{c} |
| | | | (per 100 | | | (per | | | (per |
| | n (%) | | PY) | n (%) | | 100 PY) | n (%) | | 100 PY) |
| Any AE | 522 | (93.2) | 33.3 | 338 | (94.4) | 32.9 | 318 | (88.3) | 54.2 |
| Any AE with outcome of death | 10 | (1.8) | 0.6 | 5 | (1.4) | 0.5 | 2 | (0.6) | 0.3 |
| Any AE leading to treatment | 59 | (10.5) | 3.8 | 35 | (9.8) | 3.4 | 30 | (8.3) | 5.1 |
| discontinuation | | | | | | | | | |
| Any AE of severe intensity | 102 | (18.2) | 6.5 | 67 | (18.7) | 6.5 | 46 | (12.8) | 7.8 |
| Any death of COVID-19 infection | 2 | (0.4) | 0.1 | 1 | (0.3) | 0.1 | 0 | | 0.0 |
| Any SAE (including events with | 147 | (26.3) | 9.4 | 92 | (25.7) | 9.0 | 91 | (25.3) | 15.5 |
| outcome of death) | 180 | (32.1) | | | | | | | |
| Any AESI | | | 11.5 | 113 | (31.6) | 11.0 | 61 | (16.9) | 10.4 |
| Any AESI of herpes zoster | 75 | (13.4) | 4.8 | 45 | (12.6) | 4.4 | 13 | (3.6) | 2.2 |
| Any AESI of non-opportunistic | 55 | (9.8) | 3.5 | 37 | (10.3) | 3.6 | 29 | (8.1) | 4.9 |
| serious infections | | | | | | | | | |
| Non-opportunistic serious infections | 9 | (1.6) | 0.6 | 6 | (1.7) | 0.6 | 0 | | 0.0 |
| of COVID-19 | | | | | | | | | |
| Any AESI of influenza | 36 | (6.4) | 2.3 | 20 | (5.6) | 1.9 | 11 | (3.1) | 1.9 |
| Any AESI of latent tuberculosis^{d} | 27 | (4.8) | 1.7 | 20 | (5.6) | 1.9 | 4 | (1.1) | 0.7 |
| Any AESI of opportunistic infections | 3 | (0.5) | 0.2 | 1 | (0.3) | 0.1 | 4 | (1.1) | 0.7 |
| Any AESI of anaphylaxis | 1 | (0.2) | 0.1 | 0 | | 0.0 | 0 | | 0.0 |
| Any AESI of malignancy | 12 | (2.1) | 0.8 | 7 | (2.0) | 0.7 | 4 | (1.1) | 0.7 |
| Any AESI of major acute CV | 12 | (2.1) | 0.8 | 6 | (1.7) | 0.6 | 3 | (0.8) | 0.5 |

| | | | | | | |
|---|---|---|---|---|---|---|
| events, including MI | 0 | | | | | |
| Any AESI of vasculitis | | 0.0 | 0 | 0.0 | 0 | 0.0 |

AE, adverse event;
AESI, adverse event of special interest;
COVID-19, coronavirus disease 2019;
CV, cardiovascular;
EAIR, exposure-adjusted incidence rate;
IFN, interferon;
MI, myocardial infarction;
PY, patient-years;
SAE, serious adverse event;
SLE, systemic lupus erythematosus
^{a}Data presented are combined from the TULIP trials and the extension study.
^{b}Exposure in days for each patient was calculated as the earlier of either (date of last dose of treatment + 84 days, or date of study discontinuation) − date of first dose of treatment + 1 day.
^{c}The EAIR per 100 patient-years is defined as the number of patients with the specific event divided by the total exposure in years × 100. The exposure time is defined as from the date of first administration of treatment to death, end of treatment plus 84 days, or end of study, whatever comes first.
^{d}Latent tuberculosis was defined as a positive IFN-gamma-release assay. No active cases of tuberculosis were reported.

The most common AEs in the LTE by EAIRs were nasopharyngitis (9.7 vs 5.5), urinary tract infection (8.5 vs 6.3) and upper respiratory tract infection (8.3 vs 7.2) in the LTE anifrolumab 300 mg and LTE placebo groups, respectively (Table 9). In the same groups, the most frequently reported types of SAEs by EAIRs were in the system organ class of infections and infestations (4.3 and 4.7). The EAIR of any AE with the outcome of death was 0.4 in both the LTE anifrolumab 300 mg and the LTE placebo groups, including three deaths of infections (one COVID and two pneumonia) reported with anifrolumab and one death of a major acute cardiovascular event (acute myocardial infarction) reported with placebo (Table 7). There were 2 additional deaths during the LTE study due to COVID in the group that switched from placebo to anifrolumab. When considering all anifrolumab and placebo exposure across the four years, there was a total of 12 deaths, including the 3 previously reported in the TULIP trials and the six mentioned above that occurred during the LTE. Ten of these deaths were in the all anifrolumab group, and 2 deaths were reported in the all placebo group, giving EAIRs of 0.6 vs 0.3, respectively (Table 8). Among patients in the LTE anifrolumab 300 mg and LTE placebo groups who discontinued treatment because of an AE, no DAE was reported in more than two patients per group (Table 10).

In the extension study, rates for AESIs were low in both the LTE anifrolumab 300 mg and the LTE placebo groups (Table 7). The AESI rates of non-opportunistic serious infections (3.7 vs 3.6) were similar between treatment groups. Rates of herpes zoster (HZ; 3.4 vs 2.8), latent tuberculosis (2.3 vs 0.8), and influenza (2.2 vs 0.8) were numerically higher in the LTE anifrolumab 300 mg group than in the LTE placebo group (Table 7). Of note, latent tuberculosis was defined as a positive IFN-gamma-release assay. There were no cases of active tuberculosis in either treatment group, and there were no opportunistic infections reported in the LTE anifrolumab 300 mg group. When considering the TULIP and LTE periods together, the total AESI rates of HZ decreased over time in the all anifrolumab group and were lower during the LTE period than during the first year of treatment in the TULIP trials (exposure-adjusted event rates based on time at risk: 6.8 [Year 1], 5.7 [Year 2], 3.9 [Year 3], and 2.9 [Year 4]).

To be comprehensive and include the greatest exposure, rare AEs were reported over the 4-year exposure period (all anifrolumab: n=560, total exposure 1,568.0 patient years; all placebo: n=360, total exposure 587.1 patient years). A numerically lower rate of opportunistic infections (0.2 vs 0.7) was observed in the all anifrolumab group compared with the all placebo group (0.2 vs 0.7) (Table 8). Comparably low rates of malignancy (0.8 vs 0.7), anaphylaxis (0.1 vs 0.0), and major acute cardiovascular events (0.8 vs 0.5) were observed in both treatment groups. Only non-melanoma skin cancers were reported in more than one patient in this study: basal cell carcinoma and squamous cell carcinoma (n=2, each). When considering the TULIP and LTE periods together, the rates of any SAEs among patients treated with anifrolumab (all anifrolumab group) decreased over time and were lower during the LTE than during the first year of treatment in the TULIP trials (5, 6) (n [%], exposure-adjusted event rates based on time at risk: Year 1, 53 [11.8%], 13.1; Year 2, 49 [11.3%], 12.5; Year 3, 23 [5.9%], 6.4; and Year 4, 19 [5.6%], 6.1).

Treatment-emergent anti-drug antibodies were detected in 2.6% (9/358) of patients receiving combined anifrolumab 300 mg throughout the 4-year treatment period, with no trend or pattern to suggest any association with AEs.

TABLE 9
Adverse events and event rates during treatment in the LTE, by preferred term

| | LTE Anifrolumab 300 mg | | | LTE Placebo | | |
|---|---|---|---|---|---|---|
| | (n = 257) | | | (n = 112) | | |
| | Exposure^{a} | | | | | |
| | 648.6 years | | | 236.3 years | | |
| | | | EAIR^{b} (per | | | EAIR^{b} (per |
| | n (%) | | 100 PY) | n (%) | | 100 PY) |
| Any adverse event | 225 | (87.5) | 34.7 | 91 | (81.3) | 38.5 |
| Nasopharyngitis | 63 | (24.5) | 9.7 | 13 | (11.6) | 5.5 |
| Urinary tract infection | 55 | (21.4) | 8.5 | 15 | (13.4) | 6.3 |
| Upper respiratory tract infection | 54 | (21.0) | 8.3 | 17 | (15.2) | 7.2 |
| Bronchitis | 39 | (15.2) | 6.0 | 8 | (7.1) | 3.4 |
| Headache | 27 | (10.5) | 4.2 | 11 | (9.8) | 4.7 |
| Sinusitis | 23 | (8.9) | 3.5 | 3 | (2.7) | 1.3 |
| Herpes zoster | 20 | (7.8) | 3.1 | 7 | (6.3) | 3.0 |
| Pharyngitis | 20 | (7.8) | 3.1 | 5 | (4.5) | 2.1 |
| Arthralgia | 19 | (7.4) | 2.9 | 9 | (8.0) | 3.8 |
| Oral herpes | 19 | (7.4) | 2.9 | 5 | (4.5) | 2.1 |
| Back pain | 18 | (7.0) | 2.8 | 9 | (8.0) | 3.8 |
| Cough | 18 | (7.0) | 2.8 | 3 | (2.7) | 1.3 |
| Diarrhea | 18 | (7.0) | 2.8 | 6 | (5.4) | 2.5 |
| Infusion-related reaction | 17 | (6.6) | 2.6 | 6 | (5.4) | 2.5 |

EAIR, exposure-adjusted incidence rate; LTE, long-term extension; PY, patient-years.
^{a}The exposure time is defined as from the date of first administration of treatment to the date of first event, death, end of treatment plus 84 days, or end of study, whichever comes first.
^{b}The EAIR per 100 patient-years is defined as the number of patients with the specific event divided by the total exposure time during the LTE in days for all subjects in the analysis set, multiplied by 36,525.

TABLE 10
Adverse events leading to discontinuation of the investigational product during
treatment and follow-up in the LTE and event rates by preferred term^{a}

| | LTE Anifrolumab 300 mg | | LTE Placebo | |
|---|---|---|---|---|
| | (n = 257) | | (n = 112) | |
| | Exposure^{b} | | | |
| | 683.5 years | | 250.3 years | |
| | | EAIR^{c} (per | | EAIR^{c} (per |
| | n (%) | 100 PY) | n (%) | 100 PY) |
| Any DAE | 17 (6.6) | 2.5 | 8 (7.1) | 3.2 |
| COVID-19 | 2 (0.8) | 0.3 | 1 (0.9) | 0.4 |
| Pneumonia | 2 (0.8) | 0.3 | 0 | 0.0 |
| Post herpetic neuralgia | 2 (0.8) | 0.3 | 0 | 0.0 |
| Systemic lupus erythematosus | 2 (0.8) | 0.3 | 2 (1.8) | 0.8 |
| Abortion spontaneous | 1 (0.4) | 0.1 | 0 | 0.0 |
| Acute myocardial infarction | 1 (0.4) | 0.1 | 0 | 0.0 |
| Cardiac failure chronic | 1 (0.4) | 0.1 | 0 | 0.0 |
| Chronic kidney disease | 1 (0.4) | 0.1 | 0 | 0.0 |
| Herpes zoster | 1 (0.4) | 0.1 | 0 | 0.0 |
| Infusion-related reaction | 1 (0.4) | 0.1 | 0 | 0.0 |
| Irritable bowel syndrome | 1 (0.4) | 0.1 | 0 | 0.0 |
| Latent tuberculosis | 1 (0.4) | 0.1 | 0 | 0.0 |
| Pleural effusion | 1 (0.4) | 0.1 | 0 | 0.0 |
| Renal impairment | 1 (0.4) | 0.1 | 0 | 0.0 |

AE, adverse event; COVID-19, coronavirus disease 2019; DAE, adverse event leading to discontinuation of investigational product; EAIR, exposure-adjusted incidence rate; LTE, long-term extension study; PY, patient-years.
^{a}Multiple occurrences of an AE of a preferred term in a patient are counted only once.
^{b}The exposure time is defined as from the date of first administration of treatment to the date of first event, death, end of treatment plus 84 days, or end of study, whichever comes first.
^{c}The EAIR per 100 patient-years is defined as the number of patients with the specific event divided by the total exposure time during the LTE in days for all subjects in the analysis set, multiplied by 36,525.

### COVID-19

The unprecedented global COVID-19 pandemic was declared during the last year of the LTE study which continued through the introduction of vaccines. Among patients who were in the LTE at the start of the COVID-19 pandemic in the all anifrolumab group (n=325) and the LTE placebo group (n=64), total treatment exposure was 227.7 patient years and 42.7 patient years, respectively (Table 11).

TABLE 11
COVID-19-related AEs and event rates during treatment and follow-up of the extension study

| | Number of patients ongoing at the start of the pandemic^{a} | | | | | | | | |
|---|---|---|---|---|---|---|---|---|---|
| | All Anifrolumab | | | LTE Anifrolumab | | | LTE Placebo | | |
| | (n = 325) | | | 300 mg (n = 201) | | | (n = 64) | | |
| | Total exposure during the COVID-19 pandemic^{b}, patient years | | | | | | | | |
| | 227.7 | | | 143.5 | | | 42.7 | | |
| | | | AE with | | | AE with | | | AE with |
| | | | outcome | | | outcome | | | outcome |
| | AE | SAE | of death | AE | SAE | of death | AE | SAE | of death |
| Patients with an | 33 (10.2) | 16 (4.9) | 3 (0.9) | 22 (10.9) | 10 (5.0) | 1 (0.5) | 4 (6.3) | 1 (1.6) | 0 |
| event, n (%) | | | | | | | | | |
| Time at risk^{c} | 213.6 | 221.6 | 227.4 | 134.1 | 140.4 | 143.4 | 40.7 | 42.4 | 42.7 |
| Event rate^{d} | 15.5 | 7.2 | 1.3 | 16.4 | 7.1 | 0.7 | 9.8 | 2.4 | 0.0 |
| (95% CI) | (10.6-21.7) | (4.1-11.7) | (0.3-3.9) | (10.3-24.8) | (3.4-13.1) | (0.02-3.9) | (2.7-25.1) | (0.1-13.1) | (0.00-8.6) |

AE, adverse event;
CI, confidence interval;
COVID-19, coronavirus disease 2019;
LTE, long-term extension;
SAE, serious adverse event.
^{a}The start date of the COVID-19 pandemic was Mar. 11, 2020, as declared by the World Health Organization.
^{b}Exposure during the pandemic for each patient is calculated as end of period − start date of the COVID-19 pandemic + 1 (where end of period is the earlier of either (date of last dose of treatment + 84 days, date of study discontinuation, death, or withdrawal of consent).
^{c}Total exposure for n (%) data. The time at risk is defined as time (including start and end date) from the start date of the COVID-19 pandemic to the date of first event or end of period, whatever comes first.
^{d}The event rate per 100 patient-years is defined as the number of patients with an event divided by the total time at risk during the pandemic in years × 100. Time at risk is defined as from the date of start of the pandemic to death, end of treatment plus 84 days, or end of study, whatever comes first. When reporting events occurring during treatment only, end of period plus 28 days instead of 84 days is considered

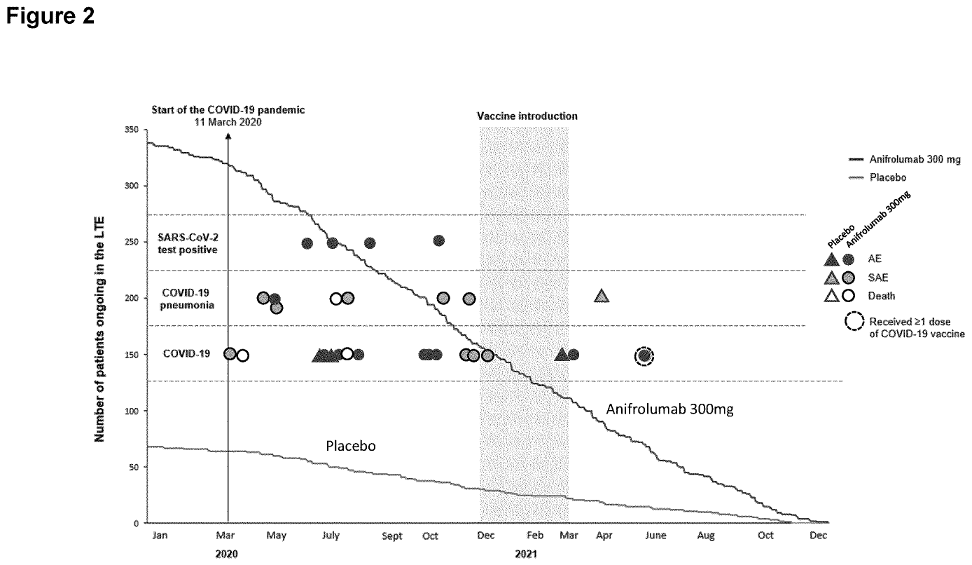

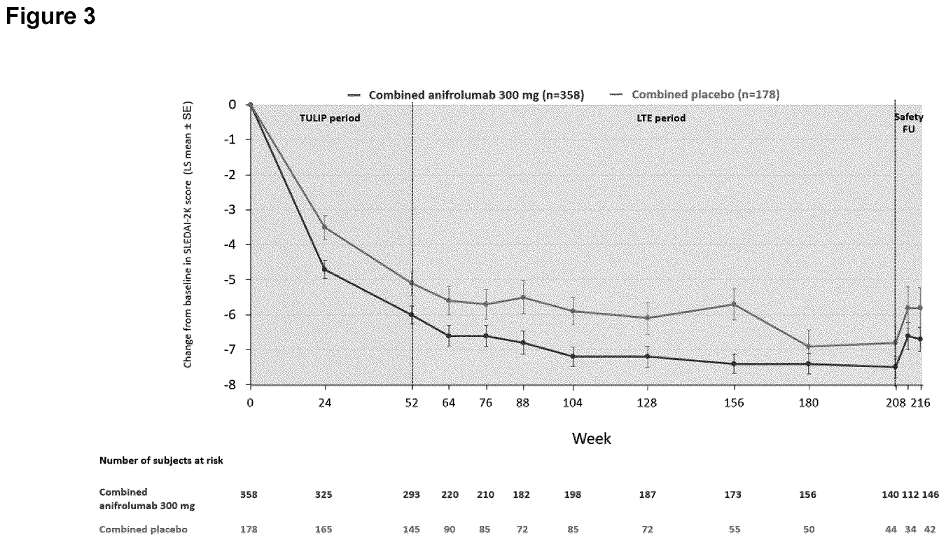

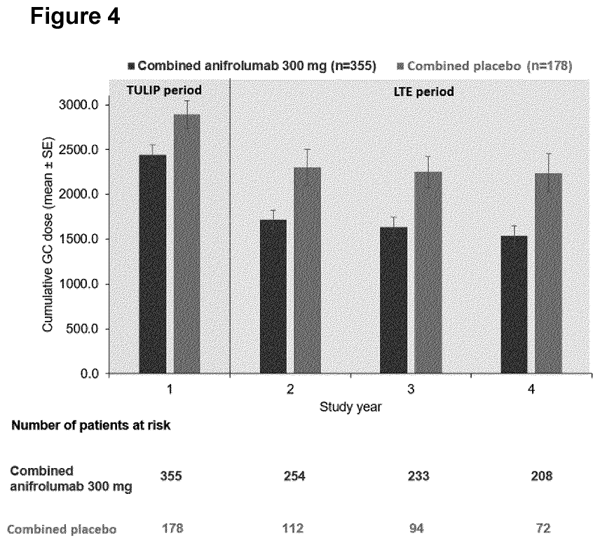

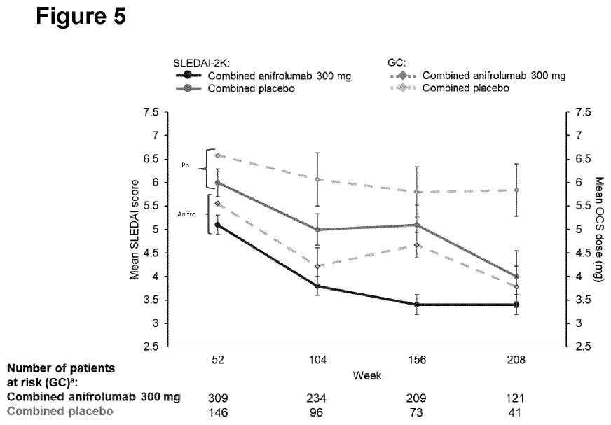

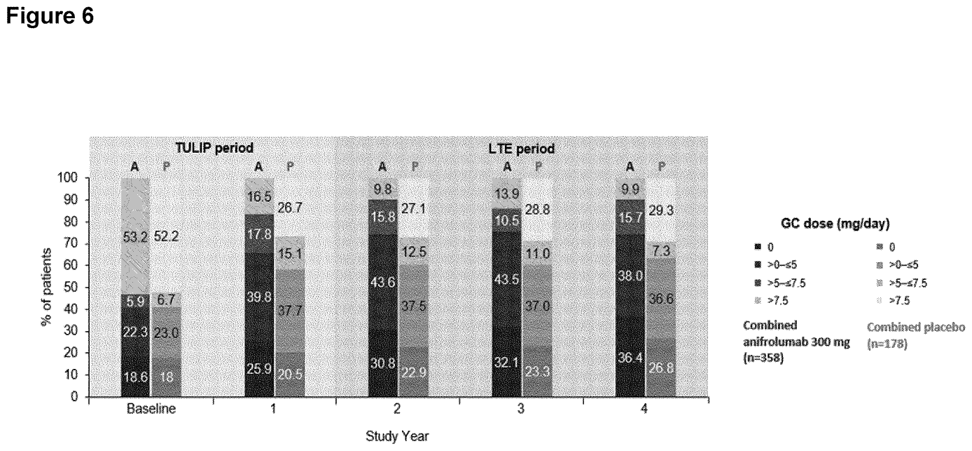

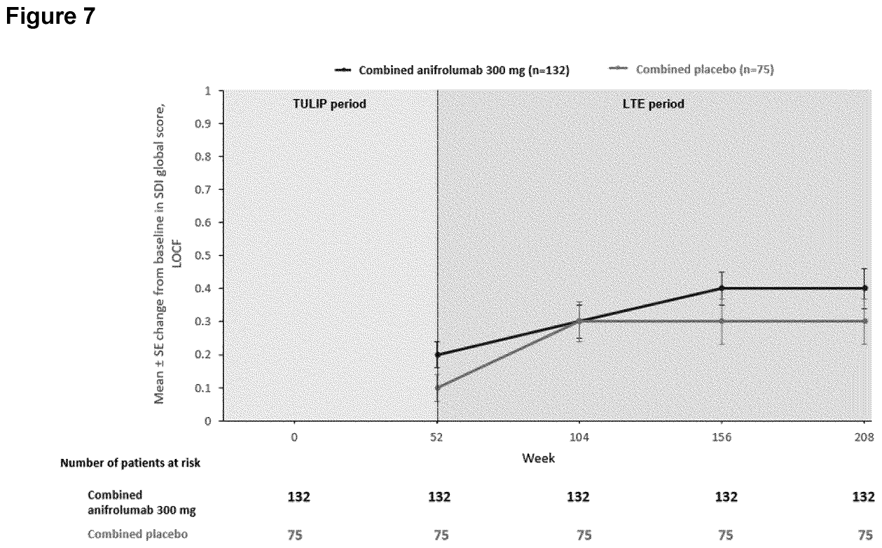

Event rates (based on time at risk during the pandemic) for COVID-related AEs and SAEs were higher in the all anifrolumab group compared with the LTE placebo group during the extension study (AEs: 15.5 vs 9.8; SAEs: 7.2 vs 2.4, respectively) (Table 11). Of the 33 COVID-related AEs in the all anifrolumab group, 16 were serious (16/33; [48.5%]); one SAE was reported in the LTE placebo group (1/4; 25%). Focusing on overall COVID-AE severity, the rate of COVID AEs was the same frequency for mild, moderate, or severe cases in the all anifrolumab group, and rates were higher compared with those reported in the broader all placebo group. Of note, asymptomatic positive COVID tests were considered COVID-related AEs. The 3 deaths in the all anifrolumab group occurred within the first 6 months of the pandemic (FIG. 2). No COVID AEs occurred in patients after they were fully vaccinated. Twenty-one patients were fully vaccinated against COVID in the anifrolumab group and nine patients were fully vaccinated in the placebo group.

### Efficacy

Efficacy data (SLEDAI-2K, PGA, glucocorticoid use, flare rates and SDI) were evaluated in the combined anifrolumab 300 mg group (n=358) versus the combined placebo group (n=178) throughout the TULIP and extension periods. For patients who continued into the LTE, the baseline (from the start of TULIP) mean (SD) SLEDAI-2K scores were 11.4 (3.8) in the combined anifrolumab 300 mg group and 11.3 (3.9) in the combined placebo group. At the start of the LTE (Week 52), mean (SD) scores were 5.1 (3.5) and 6.0 (4.1) in the two groups, respectively. During the LTE period, patients in the combined anifrolumab 300 mg group had greater mean improvement in SLEDAI-2K, with sustained improvement over time compared with the combined placebo group (FIG. 3); mean SLEDAI-2K score steadily decreased from baseline to Week 52 in the TULIP period and continued to decrease during the LTE to Week 208 in the combined anifrolumab 300 mg group.

Mean PGA score decreased from 1.8 at Week 0 (TULIP baseline) to 0.6 at Week 208 in the combined anifrolumab 300 mg group and from 1.8 at TULIP baseline to 0.7 at Week 208 in the combined placebo group (Table 12).

TABLE 12
Change in mean PGA score from baseline to Week 208^{a}

| | Combined Anifrolumab 300 mg | Combined Placebo |
|---|---|---|
| | (n = 358) | (n = 178) |
| Timepoint | Change from baseline in PGA score, mean (SD) | |
| Week 0b | 1.8 (0.4) | 1.8 (0.4) |
| Week 24 | −0.9 (0.6) | −0.7 (0.6) |
| Week 52 | −1.1 (0.7) | −0.9 (0.6) |
| Week 64 | −1.1 (0.6) | −0.8 (0.6) |
| Week 76 | −1.1 (0.6) | −0.9 (0.6) |
| Week 88 | −1.1 (0.6) | −1.0 (0.6) |
| Week 104 | −1.2 (0.6) | −0.9 (0.7) |
| Week 128 | −1.2 (0.6) | −1.0 (0.7) |
| Week 156 | −1.2 (0.6) | −1.0 (0.7) |
| Week 180 | −1.2 (0.6) | −1.2 (0.7) |
| Week 208 | −1.2 (0.6) | −1.1 (0.7) |

PGA, Physician's Global Assessment; SD, standard deviation.
^{a}Changes in PGA score presented for patients randomized to receive anifrolumab 300 mg throughout TULIP-1, TULIP-2, and the extension study (“combined anifrolumab 300 mg”) and all patients randomized to placebo in TULIP-1, TULIP-2, and the extension study (“combined placebo”).
^{b}Data are exact mean (SD) for PGA score at Week 0 (TULIP baseline).

At each of the four years during the TULIP and LTE periods, cumulative glucocorticoid dose was lower among patients in the combined anifrolumab 300 mg group compared with the combined placebo group (FIG. 4; standardized AUC), which excluded 3 patients in the anifrolumab group due to missing data. The reduction in SLEDAI-2K was achieved in parallel with reduction of the mean glucocorticoid dose (FIG. 5). The proportion of patients receiving mean glucocorticoid doses >7.5 mg/day was lower in the combined anifrolumab 300 mg group compared with the combined placebo group over the 4 years spanning the TULIP and extension periods (FIG. 6). At the end of the extension study (Year 4), 9.9% of patients in the combined anifrolumab 300 mg group were taking >7.5 mg/day of glucocorticoids compared with 29.3% in the combined placebo group (FIG. 6).

The overall annualized flare rate was 0.1 in the combined anifrolumab 300 mg group and 0.2 in the combined placebo group. All flares in both groups were mild to moderate in severity.

Mean change from baseline in SDI global score was evaluated in patients who had an SDI global score ≥1 at Week 52, including 132 patients from the combined anifrolumab 300 mg group and 75 patients from the combined placebo group (FIG. 7). At TULIP baseline, mean SDI global scores were 1.6 in the combined anifrolumab 300 mg group and 1.4 in the combined placebo group. At Week 52 (the start of the extension study), mean SDI scores were 1.8 and 1.5, respectively. During the LTE (Week 52 to Week 208), there was no difference in damage, as mean SDI score remained stable in both treatment groups (FIG. 7). A longer time (mean [SD]) to first SDI worsening was seen in the combined anifrolumab 300 mg group (925.0 [553.0] days) compared with the combined placebo group (754.2 [523.3] days).

### Discussion

Anifrolumab is a first-in-class therapy recently approved for the treatment of moderate to severe SLE despite standard of care, and the long-term data reported here are important for prescribing physicians treating patients with this chronic disease. In particular, these data described the first long-term placebo-controlled study in SLE and additionally captured the period of the global COVID pandemic. This study builds on existing evidence from the MUSE open-label extension study and shows that treatment with anifrolumab was well tolerated and has an acceptable long-term safety profile while maintaining reductions in disease activity and glucocorticoid usage.

The overall incidence of SAEs in Year 2 with anifrolumab treatment was consistent with observations during the TULIP studies, and rates decreased overtime in the LTE. The rates of both of non-opportunistic serious infections and opportunistic infections were low and comparable with placebo in this LTE. Of particular interest, the increased rate of HZ reactivation reported in the TULIP studies in the context of type I interferon blockade was further assessed here. Although there was a numerically higher rate of HZ in those treated with anifrolumab 300 mg compared with placebo during the 3-year extension period (3.4 vs 2.8), the rate with anifrolumab observed in this study was lower than that observed in pooled anifrolumab 300 mg results from the TULIP-1, TULIP-2 and MUSE studies (LTE: 3.4 vs pooled phase 2 and 3 data: 6.9). This suggests that the risk of HZ with anifrolumab is highest during the first year of treatment. The majority of HZ cases in the LTE study were mild to moderate, responded to treatment with antivirals, and all except one patient were able to continue in the study. Moreover, the overall frequency of HZ incidence with anifrolumab in the LTE was similar at 8.9% (LTE anifrolumab 300 mg: 23/257) to that reported with long-term treatment with belimumab (8.6%, 63/735) in an extension of the phase 3 BLISS-52 and BLISS-76 clinical trials in SLE [21].

Latent tuberculosis in this study was defined as a positive IFN-gamma release assay, which can lead to indeterminate results in patients with active SLE due to the use of immunomodulatory therapy and/or the common occurrence of lymphopenia. Although there were higher rates of latent tuberculosis reported in the anifrolumab group compared with placebo, perhaps due to improved disease control in the anifrolumab group, there were no cases of active tuberculosis. Our experience suggests that screening and treating latent tuberculosis is effective in the setting of anifrolumab usage.

This is the only placebo-controlled study in patients with SLE to report continuous data spanning the start (Mar. 11, 2020) [22] as well as the pre- and post-vaccination periods of the COVID-19 pandemic. There were no COVID-related AEs in patients after they were fully vaccinated against COVID. Studies have found that patients with SLE have a higher risk of severe COVID infection and poorer outcomes compared with the general population, and there have been concerns that some treatments for SLE could increase the risk of more severe viral disease [23]. Type I IFN is elevated in most patients with SLE and may play a role in the disease course of COVID [24]. Further, type I IFN responses are important in host viral defense, and their absence (ie, auto-antibody states) have been associated with high risk of severe COVID and death[26]. In the present study of long-term treatment with anifrolumab, rates of COVID-related AEs and SAEs were higher in patients treated with anifrolumab compared with placebo. Approximately 50% of the COVID-related AEs were serious in the all anifrolumab group and 25% of cases were serious in the LTE placebo group. However, most of the COVID-related SAEs and all 3 deaths occurred during the first 6 months of the pandemic, prior to the availability of vaccinations or treatments for COVID [27]. There were no COVID-related AEs in patients after they were fully vaccinated against COVID, suggesting that therapy with a IFNAR1 inhibitor surprisingly does not impact vaccine efficacy. The overall COVID mortality rate observed with anifrolumab treatment was comparable with that reported for patients with SLE treated with biologics at the time of COVID diagnosis [30]. These data show the continued efficacy of COVID vaccination in patients also treated with anifrolumab.

Malignancy rates in this study were low and comparable with placebo. Only non-melanoma skin cancers (basal cell carcinoma and squamous cell carcinoma) were reported in more than one patient in this study. Long-term malignancy risk will be further evaluated in a planned post-authorization safety study. Major cardiovascular events adjudicated by an independent cardiovascular adjudication committee were infrequent and comparable between anifrolumab and placebo. No patients experienced anaphylaxis in the LTE study.

Even though this study was primarily designed to assess long-term safety of anifrolumab in SLE, disease activity was assessed with SLEDAI-2K, and greater mean improvement was observed for patients in the combined anifrolumab 300 mg group than for patients in the combined placebo group. Improvement was sustained over time, and the mean disease activity levels remained low with anifrolumab throughout the study. Treatment with anifrolumab 300 mg enabled glucocorticoid dose reduction and lower cumulative glucocorticoid dose compared with placebo, even though there was no glucocorticoid taper required by the extension study protocol. At Year 4, 36.4% of anifrolumab-treated patients were free of glucocorticoid use (0 mg/day) and that number increased to 74.4% of patients with a glucocorticoid dose 0 or 0-55 mg/day, which may have resulted in a lower prevalence of AEs. The importance of glucocorticoid reduction in SLE has been highlighted by a recent large meta-analysis showing a clear association of glucocorticoid exposure with osteonecrosis, cardiovascular events, and osteoporosis with fractures [31]. These results in the LTE study were consistent with findings in a post hoc analysis of the TULIP phase 3 trials that showed anifrolumab facilitates sustained glucocorticoid tapering in patients with SLE and is associated with fewer SAEs. No signal for damage accrual, as measured by SDI scores, was observed in the study.

This study is unique in that it is the first phase 3, double-blind, placebo-controlled long-term safety study in SLE and it was conducted both before and during the COVID pandemic. This not only enabled initial observations of COVID infections in patients with SLE in both pre- and post-vaccination periods but also led to widespread study disruption, with interruptions to treatment in the early part of the pandemic as well as dropouts.

### REFERENCES

[1] F. Zhang et al., RMD Open 8, e001669 (2022).
[2] Y. Tanaka et al., RMD Open 7, e001629 (2021).
[3] S. Galmiche et al., Clin. Microbiol. Infect. 28, 163 (2022).
[4] K. A. Kirou et al., Front. Immunol. 13, (2022).
[5] R. Spiera, S. Jinich, and D. Jannat-Khah, Ann. Rheum. Dis. 80, 1357 (2021).
[6] D. Gladman et al., Arthritis Rheum. 39, 363 (1996).
[7] J. Albrecht et al., J. Invest. Dermatol. 125, 889 (2005).
[8] C.-S. Yee et al., Rheumatol. Oxf. Engl. 49, 1665 (2010).
[9] C.-S. Yee et al., Arthritis Rheum. 56, 4113 (2007).
[10] M. C. Hochberg, Arthritis Rheum. 40, 1725 (1997).
[11] M. Aringer et al., Arthritis Rheumatol. Hoboken NJ 71, 1400 (2019).
[12] R. Tummala et al., Lupus Sci. Med. 5, e000252 (2018).
[13] R. Furie et al., Arthritis Rheumatol. Hoboken Nj 69, 376 (2017).
[14] R. A. Furie et al., Lancet Rheumatol. 1, e208 (2019).
[15] E. F. Morand et al., N. Engl. J. Med. 382, 211 (2020).
[16] Y. Tanaka and R. Tummala, Mod. Rheumatol. 0, 1 (2020).
[17] ACR Meeting Abstracts (n.d.).
[18] A. Kuhn et al., Dtsch. Arztebl. Int. 112, 423 (2015).
[19] E. F. Morand et al., N. Engl. J. Med. 382, 211 (2020).
[20] R. A. Furie et al., Lancet Rheumatol. 1, e208 (2019).
[21] R. F. van Vollenhoven et al., Rheumatol. Oxf. Engl. 59, 281 (2020).
[22] (n.d.).
[23] M. Aringer et al., Ther. Adv. Musculoskelet. Dis. 14, 1759720X221086719 (2022).
[24] R. Fernandez-Ruiz, J. L. Paredes, and T. B. Niewold, Transl. Res. J. Lab. Clin. Med. 232, 13 (2021).
[25] WHO Solidarity Trial Consortium et al., N. Engl. J. Med. 384, 497 (2021).
[26] P. Bastard et al., Science 370, eabd4585 (2020).
[27] RECOVERY Collaborative Group et al., N. Engl. J. Med. 384, 693 (2021).
[28] B. Meng et al., Nature 603, 706 (2022).
[29] R. Suzuki et al., Nature 603, 700 (2022).
[30] M. F. Ugarte-Gil et al., Ann. Rheum. Dis. 81, 970 (2022).
[31] M. F. Ugarte-Gil et al., Lupus Sci. Med. 8, e000590 (2021).

1. A method for treating systemic lupus erythematosus (SLE) and for preventing or reducing the risk of SARS-CoV-2 infection in a subject in need thereof, the method comprising
a) administering a type I IFN receptor (IFNAR1) inhibitor to the subject, and
b) administering a therapeutically effective amount of a SARS-CoV-2 vaccine to the subject,
wherein the method prevents or reduces the risk of SARS-CoV-2 infection in the subject following exposure of the subject to SARS-CoV-2, and wherein the method treats SLE in the subject.

2. The method of claim 1, wherein the IFNAR1 inhibitor is administered to the subject less than 1 month before the SARS-CoV-2 vaccine.

3. The method of claim 1 or 2, wherein the IFNAR1 inhibitor is administered on the same day as the SARS-CoV-2 vaccine.

4. The method of any of preceding claim, wherein the IFNAR1 inhibitor is administered intravenously or subcutaneously.

5. The method of any preceding claim, wherein the IFNAR1 inhibitor is administered to the subject at a dose of 120 mg to 1000 mg.

6. The method of claim 1, wherein the IFNAR1 inhibitor is administered to the subject intravenously at a dose of about 300 mg every 4 weeks (Q4W).

7. The method of claim 1, wherein the IFNAR1 inhibitor is administered to the subject intravenously at a dose of about 900 mg every 4 weeks (Q4W).

8. The method of claim 1, wherein the IFNAR1 inhibitor is administered to the subject subcutaneously at a dose of about 120 mg every week.

9. A method for preventing or reducing the risk of SARS-CoV-2 infection in a subject in need thereof, wherein the subject has an autoimmune disease and has been treated with a IFNAR1 inhibitor, the method comprising administering a therapeutically effective amount of a SARS-CoV-2 vaccine to the subject, and wherein the method prevents or reduces the risk of SARS-CoV-2 infection in the subject following exposure of the subject to SARS-CoV-2.

10. The method of claim 9, wherein administration of the SARS-CoV-2 vaccine prevents or reduces the risk of COVID-19 pneumonia in the subject.

11. The method of claim 9 or 10, wherein administration of the SARS-CoV-2 vaccine prevents or reduces the risk of COVID-19 associated death in the subject.

12. The method of any of claims 9 to 11, wherein the SARS-CoV-2 vaccine is administered to the subject 1 month or less following administration of the IFNAR1 inhibitor to the subject.

13. The method of any of claims 9 to 12, wherein the IFNAR1 inhibitor was administered intravenously or subcutaneously.

14. The method of any of claims 9 to 13, wherein the IFNAR1 inhibitor was administered to the subject at a dose of 120 mg to 1000 mg.

15. The method of any of claims 9 to 14, wherein the IFNAR1 inhibitor was administered to the subject intravenously at a dose of about 300 mg every 4 weeks (Q4W).

16. The method of any of claims 9 to 14, wherein the IFNAR1 inhibitor was administered to the subject intravenously at a dose of about 900 mg every 4 weeks (Q4W).

17. The method of any of claims 9 to 14, wherein the IFNAR1 inhibitor was administered to the subject subcutaneously at a dose of about 120 mg every week.

18. The method of any of claims 9 to 17, comprising administering at least 2 doses of the SARS-CoV-2 vaccine to the subject.

19. The method of claim 18, wherein the 2 doses of the SARS-CoV-2 vaccine are administered to the subject within a period of 21 to 28 days.

20. The method of claim 18, wherein the 2 doses of the SARS-CoV-2 vaccine are administered to the subject within a period of 14 to 56 days.

21. The method of any of claim 9 or 20, the IFNAR1 inhibitor was administered to the subject less than 1 month before the SARS-CoV-2 vaccine is administered to the subject.

22. A method for treating SLE in a subject in need thereof, wherein the subject has been vaccinated against COVID-19 with a SARS-CoV-2 vaccine, the method comprising administering a therapeutically effective amount of a IFNAR1 inhibitor to the subject, wherein the method treats SLE in the subject.

23. The method of claim 22, wherein the subject has been fully vaccinated against COVID-19.

24. The method of claim 22 or 23, wherein the subject has received at least 2 doses of the SARS-CoV-2 vaccine.

25. The method of claim 24, wherein the 2 doses of the SARS-CoV-2 vaccine were administered to the subject within a period of 21 to 28 days.

26. The method of claim 24, wherein the 2 doses of the SARS-CoV-2 vaccine were administered to the subject within a period of 14 to 56 days.

27. The method of claim 22 to 26, wherein the subject was vaccinated against COVID-19 6 months or less before administration of the IFNAR1 inhibitor.

28. The method of any preceding claim, wherein the SARS-CoV-2 vaccine is selected from the group consisting of AZD1222, mRNA-1273, or BNT162b2 Tozinameran or combinations thereof.

29. The method of any preceding claim, wherein the IFNAR1 inhibitor is a human monoclonal antibody specific for IFNAR1, optionally a modified IgG1 class human monoclonal antibody.

30. The method of claim 29, wherein the antibody comprises:
(a) a heavy chain variable region complementarity determining region 1 (HCDR1) comprising the amino acid sequence of SEQ ID NO: 3;
(b) a heavy chain variable region complementarity determining region 2 (HCDR2) comprising the amino acid sequence of SEQ ID NO: 4;
c) a heavy chain variable region complementarity determining region 3 (HCDR3) comprising the amino acid sequence of SEQ ID NO: 5;
(d) a light chain variable region complementarity determining region 1 (LCDR1) comprising the amino acid sequence SEQ ID NO: 6;
(e) a light chain variable region complementarity determining region 2 (LCDR2) comprising the amino acid sequence SEQ ID NO: 7; and/or
(f) a light chain variable region complementarity determining region 3 (LCDR3) comprising the amino acid sequence SEQ ID NO: 8.

31. The method of claim 29 or 30, wherein the antibody comprises: (a) a human heavy chain variable region comprising the amino acid sequence of SEQ ID NO: 1; and (b) a human light chain variable region comprising the amino acid sequence of SEQ ID NO: 2.

32. The method of any of claims 29 to 31, wherein the antibody comprises an Fc region comprising amino acid substitutions of L234F, L235E and/or P331S, as numbered by the EU index as set forth in Kabat.

33. The method of any of claims 29 to 32, wherein the antibody comprises: (a) a human heavy chain comprising the amino acid sequence of SEQ ID NO: 11; and (b) a human light chain comprising the amino acid sequence of SEQ ID NO: 12.

34. The method of any preceding claim, wherein the IFNAR1 inhibitor is anifrolumab or a functional variant thereof.

35. A pharmaceutical composition for use a method of preventing or reducing the risk of SARS-CoV-2 infection in a subject, wherein the subject has an autoimmune disease and has been treated with a IFNAR1 inhibitor, wherein the pharmaceutical composition comprises a SARS-CoV-2 vaccine, the method comprising administering the pharmaceutical composition to the subject, wherein the pharmaceutical composition prevents or reduces the risk of SARS-CoV-2 infection in the subject following exposure of the subject to SARS-CoV-2.

36. A pharmaceutical composition for use in a method of treating SLE in a subject in need thereof, wherein the subject has been vaccinated against COVID-19 with a SARS-CoV-2 vaccine, wherein the pharmaceutical composition comprises a IFNAR1 inhibitor, the method comprising administering the pharmaceutical composition to the subject, wherein the method treats SLE in the subject.

37. An injection device comprising the pharmaceutical composition of claim 35 or 36.

38. The injection device of claim 37, wherein the injection device is a pre-filled syringe (PFS), optionally an accessorized pre-filed syringe (AFPS), or an auto-injector.

39. A kit comprising the injection device of claim 37 or 28, and instructions for use.

40. The kit of claim 39, wherein the instructions for use comprise instructions for subcutaneous administration of the pharmaceutical composition to the subject.

41. The kit of any of claim 39 or 40, wherein the instruction for use specify that the pharmaceutical composition is for use in the method of any of claims 1 to 34.
